(19) United States
(12) Patent Application Publication
(10) Pub. No.: US 2019/0348155 A1
(43) Pub. Date: Nov. 14, 2019

(54) COORDINATED IN VITRO AND IN SILICO BASED APPROACH FOR PREDICTING NANOMATERIAL BIODISTRIBUTION

(71) Applicant: University of Central Florida Research Foundation, Inc., Orlando, FL (US)

(72) Inventors: Edward PRICE, Casselberry, FL (US); Andre GESQUIERE, Chuluota, FL (US)

(21) Appl. No.: 16/257,000

(22) Filed: Jan. 24, 2019

Related U.S. Application Data
(60) Provisional application No. 62/621,288, filed on Jan. 24, 2018.

(51) Int. Cl. G16C 20/30; G01N 21/64

(52) U.S. Cl. CPC G16C 20/30; G01N 2021/6441; G01N 21/6428

(57) ABSTRACT

Disclosed herein is a new system that eliminates the need for animal testing of nanomaterial (NM) biodistribution. The system involves a streamlined in vitro assay that is merged with in-silico approaches that enable accurate prediction of the biodistribution of a variety of NMs in the body. Due to the design of certain embodiments, unprecedented resolution is obtained, where cellular level knowledge/cellular resolution in tissues in the body is delivered for the first time.

Nanomaterial (NM) applications span across fields such as medicine, electronics, materials engineering, and agriculture. Considering the extent of this progress, exposure or ingestion of NMs has become a concern and has stifled growth due to difficulties in receiving regulatory approval for NM applications, especially in medicine and agriculture. Challenges with accurately describing NM biodistribution and behavior in the body are thus important to overcome. Increased deployment of NM applications has pushed the need for robust methodologies to properly quantify NM-biological interactions accurately and predict biodistribution in the body.

Accurate quantification of NMs at the tissue and cellular level is therefore vital and requires assessment of true tissue and cellular level dose in the body. Current studies rely on animal testing. Current processing methods for in vivo samples often require animal sacrifice, tissue resection for histological analysis and possible further processing via homogenization and/or acidic degradation, all which require tissue samples taken postmortem. This process sacrifices animals, is time-consuming, and requires substantial resources as often only one time point is available per animal. Additionally, tissue homogenization of resected tissues is destructive to cells and tissue architecture, resulting in loss of important cellular level information. Increased production of NMs continues to exacerbate these challenges. This warrants the need for a new method that can quantify NM accumulation accurately, precisely, and efficiently without animal sacrifice.

While a good degree of success has been obtained in moving away from animal testing for small molecule drugs by using computer models, these models do not apply directly to NMs.

### SUMMARY

Disclosed herein is a new system that eliminates the need for animal testing of NM biodistribution. The system involves a streamlined in vitro assay that is merged with in-silico approaches that enable accurate prediction of the biodistribution of a variety of NMs in the body. Due to the design of certain embodiments, unprecedented resolution is obtained, where cellular level knowledge/cellular resolution in tissues in the body is delivered for the first time. The system eliminates the need for animal testing, reduces cost of NM testing, and presents a predictive approach that furthers smart NM based product development. The disclosed system provides a process for in vivo whole body predictive modeling based on mechanistic biology to answer key questions about tissue accumulation and biodistribution of NM in vivo.

### BRIEF DESCRIPTION OF THE DRAWINGS

FIG. 1 shows a diagram depicting different aspects of the In Vitro Modeling and In Silico Modeling to determine NM biodistribution.

FIG. 2 shows a diagram illustrating constants associated with NM uptake by cells.

FIG. 3 shows a schematic diagram relating to an RCF assay method embodiment.

FIG. 4 shows a diagram showing basic mass transfer kinetics via first-order rate constants.

FIG. 5 shows a diagram of an NM full physiologically-based pharmacokinetic (PBPK) model.

FIGS. 6A and 6B show graphs, FIG. 6A revealing cell uptake of NM as measured and modeled with degradation (blue) and without degradation (red); and a graph FIG. 6B revealing model prediction of degradation of NM (red) into free cadmium (blue) compared to lysosomal experiments

FIG. 7A shows a diagram of analysis techniques for determining cadmium content upon degradation of NM.

FIG. 7B shows a graph relating to detection of Cadmium content.

FIG. 8 shows a diagram illustrating the relationship between vasculature pore size and NM size used to capture reflection coefficients.

FIGS. 9A, 9B, 9C and 9D show genetic algorithm convergence to the optimal combination of rate constants. FIG. 9A shows a group representing genetic algorithm determination related to PS-COOH Fitness. FIG. 9B provides a graph representing genetic algorithm determination related to QSH fitness. FIG. 9C shows PS-COOH Average Distance between individuals. FIG. 9D shows QSH average distance between individuals.

FIG. 10 shows the error observed upon model fit to RCF dataset.

FIG. 11 provides a diagram showing the relevant factors involved in determining transfer of NM from blood to interstitial and from interstitial to macrophages and epithelial cells.

FIGS. 12A, 12B, 12C, 12D and 12E show illustrations with data representative of model outputs for cell simulation. FIG. 12A shows QSH and PSCOOH model fits (line) to data (open circle) for washed cell samples taken from RCF datasets. FIG. 12B shows Model output prediction for amount of QSH and PS-COOH internalized by cells. FIG. 12C shows Model output prediction for amount of QSH and PS-COOH adhered to membrane. FIG. 12D Is representative of all kinetic rate constants optimized the genetic algorithm in MATLAB. Rate constants here include adsorption (kads), desorption (kdes), and internalization (kint). FIG. 12E shows Confocal imaging of Hepa1-6 cells exposed to 10 nM QSH and PS-COOH from RCF datasets. Hoechst33258 (blue) and QD or QSH (red) are shown above. QSH shows no saturability for all time points from 0 to 24 hours. PS-COOH shows saturable uptake around 2 hours. Error bars in (a) are descriptive of upper and lower bounds of 95% confidence interval for model fit to data.

FIGS. 13A, 13B, 13C and 13D shows schematic overviews of the overall scope for RCF method development. FIG. 13A a schematic of the mechanisms of cellular internalization. FIG. 13B shows cellular degradation and interactions with QDs. FIG. 13C shows a proof of concept simulation built from the proposed fluorometric datasets with parameters representative of adsorption, desorption and internalization pathways. FIG. 13D shows validation results.

FIG. 14 shows a bar graph representing unwashed quantum dot (QD) fluorescence data for multiple different cell lines.

FIG. 15 provides a line graph of QD uptake in J774a.1 cells as evidenced by fluorescence.

FIG. 16 provides a line graph of QD uptake in AML12 cells as evidenced by fluorescence.

FIG. 17 provides a line graph of QD uptake in MES 13 cells as evidenced by fluorescence.

FIG. 18 provides a line graph of QD uptake in C166 cells as evidenced by fluorescence.

FIG. 19 provides a graph showing calibrated and raw data of AML12 QSH Cell uptake represented by nanomolar concentration.

FIG. 20 provides a graph showing calibrated and raw data of C166 QSH Cell uptake represented by nanomolar concentration.

FIG. 21 provides a graph showing calibrated and raw data of J774a.1 QSH Cell uptake represented by nanomolar concentration.

FIG. 22 provides a graph showing calibrated and raw data of MES13 QSH Cell uptake represented by nanomolar concentration.

FIG. 23 provides confocal micrographs showing uptake of quantum dots. The table represents Rate constants related to each of the cell lines.

FIG. 24 is a table showing the statistical data for the graphs in 19-22.

FIG. 25 shows a bar graph showing nanomaterial size dependency on liver uptake.

FIG. 26 shows line graphs indicating sensitivity to changes in nanomaterial diameter with subcellular resolution (membrane vs. cellular space). FIG. 26A shows sensitivity with respect to endothelial cell membrane. FIG. 26B shows sensitivity with respect to endothelial cell space.

FIG. 26C shows sensitivity with respect to interstitial.

FIG. 27 shows a bar graph demonstrating simulation accuracy at the single cell level within splenic macrophages, with simulations falling to less than 2-fold error. When not calibrating to cellular-induced degradation, simulation falls to greater than 2-fold error, suggesting importance in including degradation effects.

FIG. 28 shows line graphs demonstrating sensitivity to size of nanomaterial in splenic macrophage membrane (FIG. 28A) and macrophage space (FIG. 28B).

FIG. 29 shows line graphs demonstrating sensitivity to size of nanomaterial in epithelial cell membrane (FIG. 29A) and epithelial cell space (FIG. 29B).

FIG. 30 shows line graphs of predicted and observed fluorescence signals in plasma, liver and kidney of rat (FIG. 30A), mouse (FIG. 30B) and primate (FIG. 30C) using different nanomaterials.

FIG. 31 shows line graphs of predicted and observed fluorescence signals in various cell types (spleen, heart, lung, brain, muscle and bone cells) for of rat (FIG. 31A), mouse (FIG. 31B) and primate using different nanomaterials (FIG. 31C).

FIG. 32 is a table showing the average fold error, predicted vs. observed in various cells types in rat, mouse, and primate using different nanomaterials. When not calibrating to cellular-induced degradation, simulation falls to greater than 2-fold error, suggesting importance in including degradation effects. [Note that the color key in FIG. 33 also applies to FIG. 32].

FIG. 33 shows a line graph of predicted vs. observed results and fold error analysis of the datasets. FIG. 33A shows a graph of predicted vs. observed results. FIG. 33B is a graph showing Fold Error Analysis. FIG. 33C is a table summarizing results of FIG. 33A and FIG. 33B.

### DETAILED DESCRIPTION

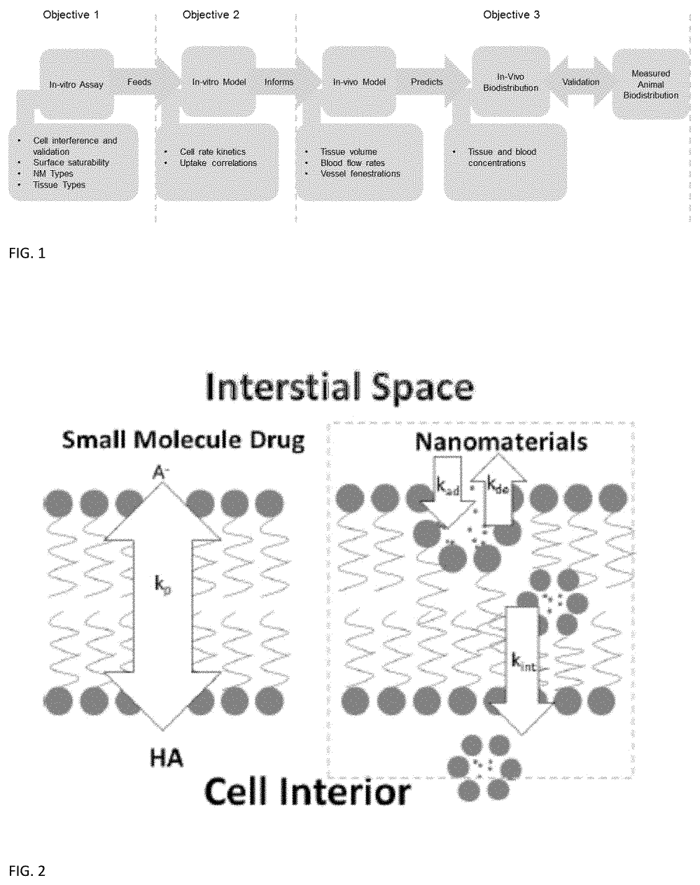

According to a primary embodiment, disclosed herein is a system outlined in FIG. 1 for assessing biodistribution of NM in vivo. A first part of the system comprises a novel high-throughput quantitative assay based on ratiometric corrected fluorescence (RCF) that takes into account effects of cell-induced and media induced loss of fluorescence from NM. The RCF measurements are used with an in vitro model to produce cell rate kinetics and uptake correlations. An assembly of the in vitro model from multiple cell types is then used to produce a mechanistic in vivo simulation model (or PBPK model) that takes into account tissue volume, cellular compositions of tissues, blood flow rates, vessel fenestrations, and kinetics of cell-NM interactions. Using the in vivo simulation model, in vivo biodistribution (tissue and blood concentrations) can be predicted across tissues with cellular resolution. The biodistribution predictions can be validated using real life animal biodistribution data.

According to a specific embodiment, a Genetic Algorithm (GA) is used to extract rate kinetics from in vitro NM-cell interactions determined by the in vitro RCF assay to deliver accurate and precise quantification of the kinetics of NM interaction with cells via physiologically relevant rate constants that translate into whole body simulations. A PBPK model that predicts the biodistribution of NMs in the body (in vivo) is produced based on these in vitro cell rate kinetics, giving the simulation a cellular level resolution inside tissues.

Embodiments disclosed herein provide cellular level resolution of NM interaction and distribution inside tissues in a whole body. This is based on the unique design of the high throughput RCF assay (in vitro), the coupling of the high throughput RCF assay to a cell kinetics simulation, the use of Genetic Algorithm (GA) in the cell kinetics simulation to extract rate kinetics from in vitro NM-cell interactions, and the use of a novel predictive whole body simulation (in vivo) that uses the GA outputs.

The predictive computer PBPK model that delivers the whole body simulation of NM biodistribution is a radical departure from current paradigm. According to embodiments disclosed herein, NM distribution in the body is based on rate kinetics, which is much different from the current approaches in which in vivo distribution is described by partitioning and immediate equilibrium in a tissue from optimization of models to animal studies. The partitioning and equilibrium approach works for small molecules because small molecules tend to diffuse into tissues rapidly, but has not provided predictive power for NMs. Also note that current NM models provide gross distribution but do not deliver information on cellular level interactions i.e. no cellular resolution. Instead, disclosed is a kinetics based description that takes into account that NMs interact with cells mainly through activated transport, not passive diffusion, and provides cellular resolution. This is important, as many drugs need to reach target tissue cells in order to be effective.

NM based models reported elsewhere are still in their infancy. Currently, they rely heavily on experimental in vivo datasets, making them descriptive rather than predictive, as well as incorporate commonly used tissue-specific partition coefficients representative of immediate equilibration between tissue and blood, or kₚ. This is another assumption that is applicable to small molecules, but NMs follow time-driven active-transport processes that require rate-driven kinetics (see FIG. 2).

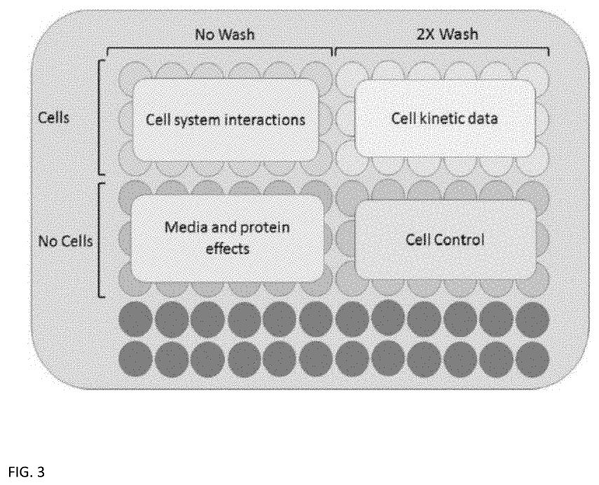

Because NMs interact with cells mainly through activated transport, not passive diffusion, using small molecule predictive models has had limited impact in the study and predictive quantification of NM in vivo biodistribution. As noted above, a kinetics based description that takes into account that NMs interact with cells mainly through activated transport is required. The embodiment as shown in FIG. 3 serves the function of quantifying these kinetics. This part of the system generates kinetics constants by determining interactions of NM with cells in vitro (such as by measuring NM generated fluorescence) and yields quantitative data on rate-driven kinetics in vitro that serves as necessary inputs for predictive in-silico in vivo NM simulations. The fluorescent determinations take into account effects of cell-induced and media induced loss of fluorescence from NM, which has been largely ignored in literature due to the difficulty in quantifying the effect. Due to the design of the assay, the Cell-NM fluorescence assay provides accurate kinetic data regardless of cell/media-induced fluorescence degradation being present or not. The problem of subcellular NM localization is solved through coupled in vitro experiments and simulations which quantify rates of NM interaction (adsorption, internalization, metabolism, desorption) with cells. In a specific embodiment, Genetic Algorithm is used to extract rate kinetics from in vitro NM/cell interactions. Results of the NM/cell fluorescence assay have been validated by benchmarking results from the assay against atomic absorption spectroscopy (AAS). AAS was chosen as validation of this fluorescence technique due to its standing as an EPA standard. The quantitative output from the assay reasonably (P>0.05) matched AAS. Confocal imaging of the endpoint of the assay further confirmed findings visually.

By obtaining RCF measurements from cell lines representative of different tissues (e.g. liver, kidneys, etc.) in vitro, rate based kinetics are extracted for each tissue type. The in vivo simulation (or PBPK) model contains each tissue type as a compartment that interacts with NMs through a rate based kinetic model, and the compartments are connected by parameters describing blood flow rate between and inside the different tissues (as shown in FIG. 5), and is comprised of multiple sets of ODEs (ordinary differential equations), each connected through mass transfer and tissue blood flow rates. The in vivo simulation model typically includes all tissues of the species being analyzed, as well as tissue sub-compartments comprised of cell membrane, cell space, and interstitial space. Each tissue has its own weight, as well as total blood flow rate (Qₓ, where x=tissue type), and number of cells of each type in the tissue (e.g. macrophage, epithelial, etc.). In addition, at the macroscopic level, all tissues are interconnected via blood flow rates that act as transport systems of the various NM types. Specifically, an arterial flow rate towards the tissue and a venous flow rate leaving the particular tissue. New parameters in this model reflect accurate biological and mechanistic assumptions for NM disposition.

Here, the parameters optimized to the in vitro datasets guide NM active transport between the cell and the surrounding environment inside the in vivo simulation model. Each tissue is compartmentalized into cellular-level architecture. For example, epithelial tissue cells (EC) including macrophages, interstitial fluid (IS), endothelial cells, and lymphatic vessels (LV) all make up an entire tissue compartment and reflect accurate physiological properties. The kinetic data for tissues and NM is incorporated as an input from the in vitro model.

In the in vivo simulation model, all paracellular transport into IS and lymph nodes (LN) is defined through tissue and size specific reflection coefficients (σ) (see FIG. 5). This allows for only a specific fraction of NM to pass into the next compartment, given the size. Reflection coefficients are smaller in value for tissues with large pores or sinusoids (e.g. liver, pancreas) and larger for tissues with minimal fenestrae (e.g. brain, kidney). Regions that contain reflection coefficients include 1) blood supply-interstitia and 2) lymphatic vessel-interstitia interfaces. Transcellular transport is primarily accounted for with the endothelial cells themselves in the blood vessel lining. Once inside the interstitial space, the NM can interact with the epithelial and macrophage cell membrane compartment through adsorption, desorption, or internalization (rates optimized from the in vitro model). Otherwise, the NM may also get taken up through the lymphatic vessels through the aforementioned reflection coefficient. Once inside the lymph vessels, the NMs drain to a central lymph compartment and back into the blood supply.

### RCF Assay and Generation of In Vitro Model

FIG. 2 shows traditional partition coefficients (kₚ) commonly used for small molecule partitioning between blood supply and cell interior. For NMs, rate constants for adsorption (k_{ad}), desorption (k_{de}), and internalization (kᵢₙₜ) more accurately represent uptake processes incident on a NM when exposed to cell environment. FIG. 3 shows the schematic overview of the RCF assay: upper left region is descriptive of cellular degradation and interactions with NMs; upper right, descriptive of fluorescence after washing cells exposed to NMs; bottom left, descriptive of biological and/or media effects on NM; bottom right, descriptive of control with cells with no NM exposure.

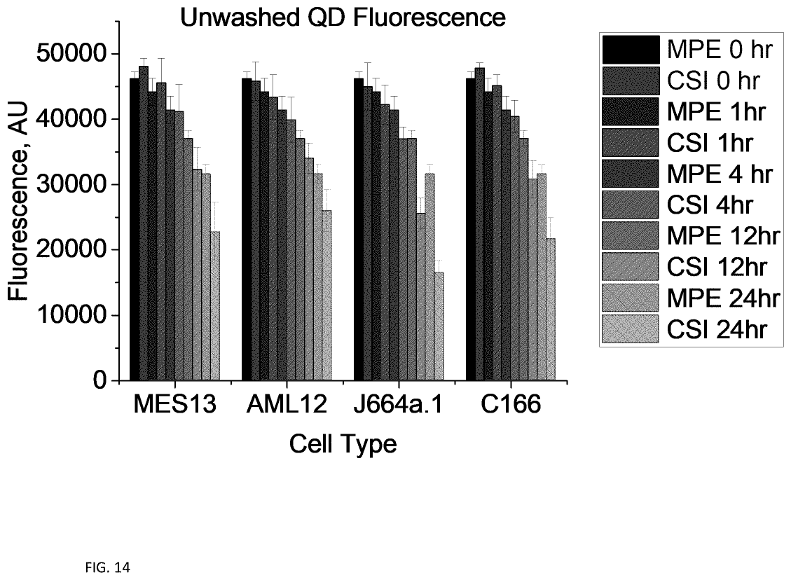

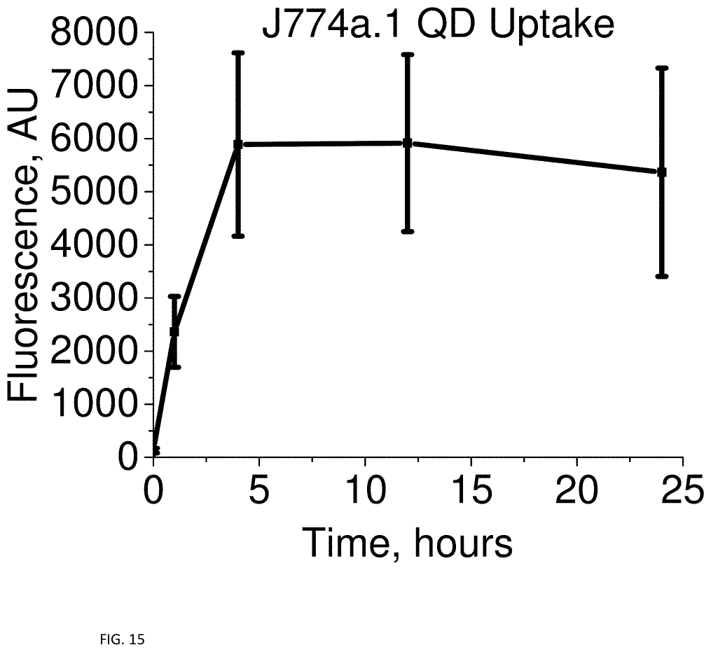

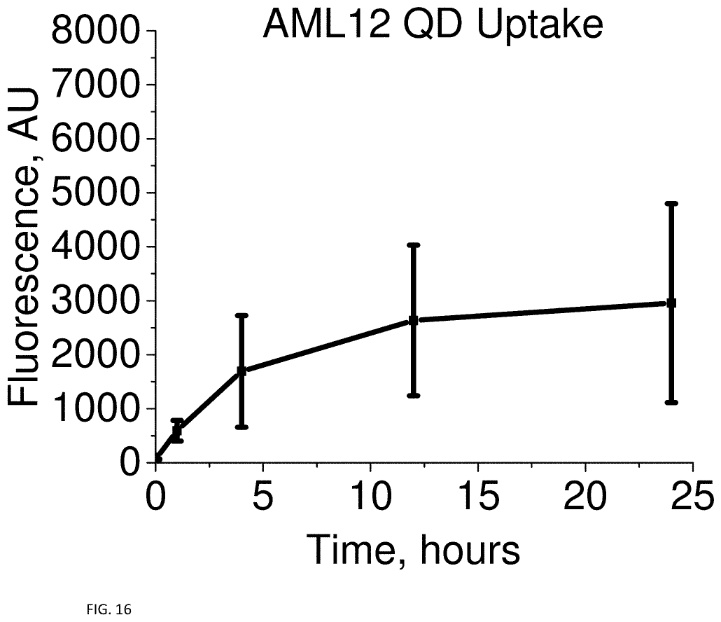

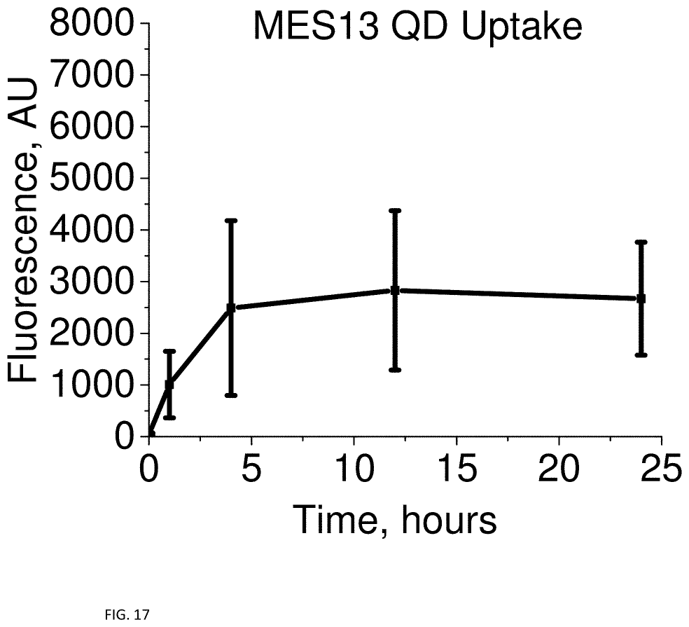

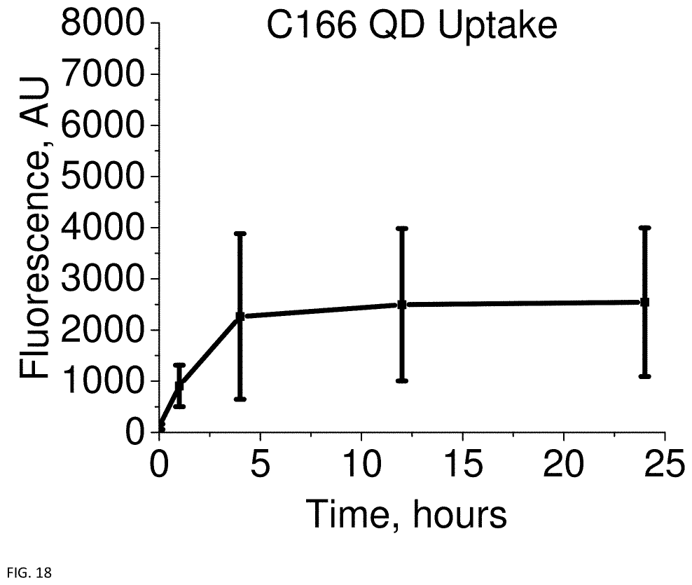

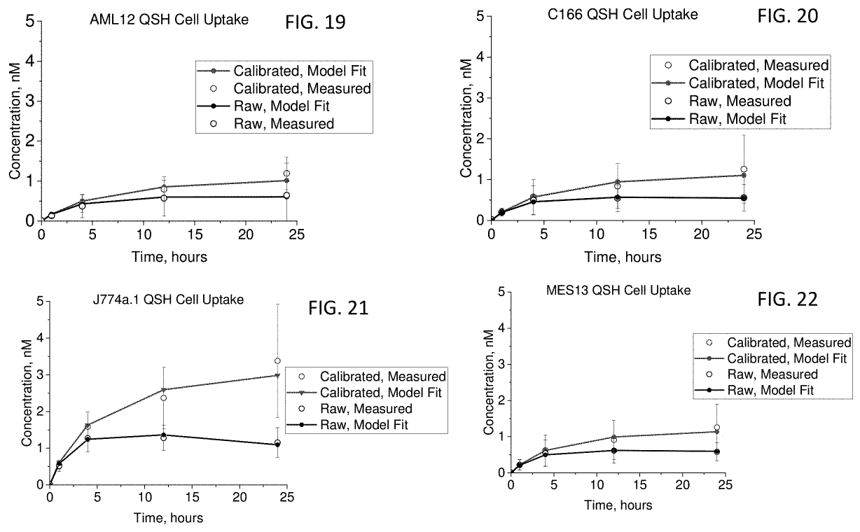

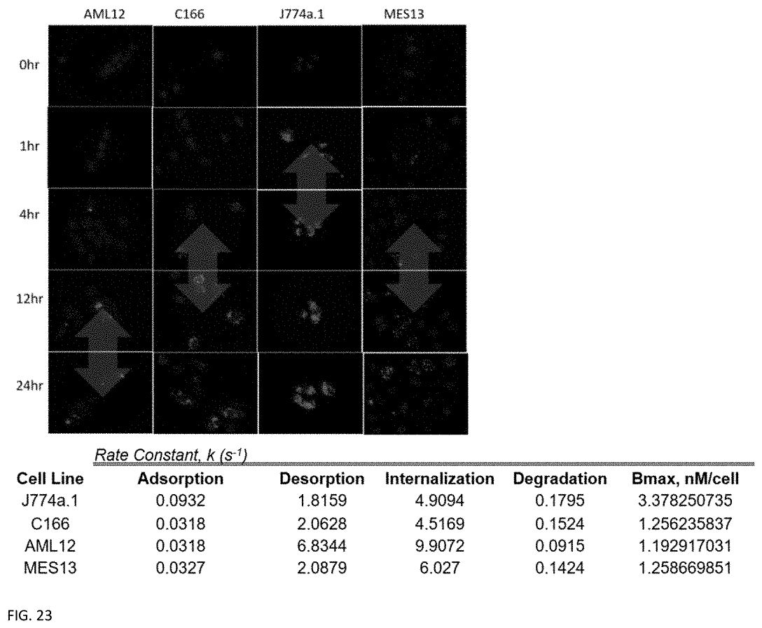

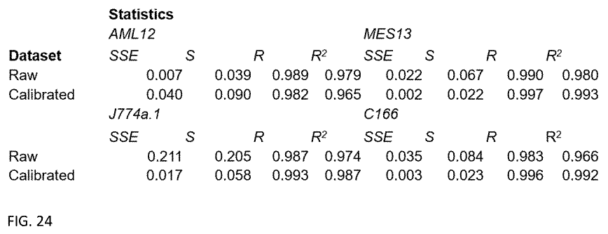

In one example as illustrated in FIG. 3, cells are applied to all columns in the first three rows on a well plate (e.g., 96-well plate system). Microscopic analysis is performed to determine optimal seeding density for cell application, starting with 100,000 cells/cm² to achieve semi-confluence after 48 hours. When cells are ready, NMs are applied to all wells in 3 groups (labeled Cell System Interactions (CSI), and Cell Kinetic Data (CKD)) in addition to the additional 3 rows with no cells (Media and Protein Effects, or MPE). The Cell Control set of wells is left alone and used as the control well with no NM application. At particular time points, wells from the CKD compartment are washed 2 times, trypsinized, and measured for their fluorescent values. FIGS. 14-18 represent graphs of fluorescent values. Fluorescent values are converted to nanomolar quantities according to equation 1 below. FIGS. 19-22 represent concentration graphs based on fluorescent values in FIGS. 14-18. In this example, fluorescent quantum dots (QDs) are used as a surrogate for NMs. [For testing nanomaterials that do not have fluorescence, these would need to be tagged with visual (e.g. fluorescence) marker.]

$${[ QD ]}_{t} = \frac{{I}_{{washed}_{t}}}{{I}_{{unwashed}_{x}}} * Dose$$

where I_{washedt} is the intensity of cells washed 2×, I_{unwashedx} is the intensity of unwashed where x=0 or t and cell or no cell exposure. If fluorescence quenching or degradation is significant, unwashed intensity at time t with cell exposure (CSI compartment) is used. If no fluorescence quenching or degradation is significant at time t, unwashed intensity with no cell exposure at time 0 is used (MPE compartment). Cell degradation or quenching is analyzed by determination of significant difference of the CSI and MPE compartments at time, t. If no cell quenching or degradation is apparent, sample set means of CSI and MPE compartments should not differ significantly (P>0.05). If degradation or quenching exists, CSI compartment fluorescence should decrease faster with time. If this is the case, the washed intensity will be divided by the unwashed intensity, shown above, to wield a corrected fractional uptake, which is then multiplied by dose in nanomolar to yield a concentration of uptake. After full analysis at all time points, cells containing QDs in this same experiment are transferred to petri dishes and stained with lysotracker green (lysosomal stain) for confocal imaging. The confocal images provide 1) qualitative and quantitative understanding of cellular uptake and 2) sequestration in lysosomes over time. Increase in QD accumulation over time as well as lysosomal sequestration is expected as exposure time is increased. At least 10 images per petri dish are taken as representative fields of view for experimental time points. Additionally, confocal images are taken with z-slices at approximately 2 um apart, totaling 40 um in diameter. Images are fully analyzed for QD uptake and quantified through increases in pixel intensities over time for the z-stack. FIG. 23 represents confocal images for the cell uptake in cells used for the data in FIGS. 14-22.

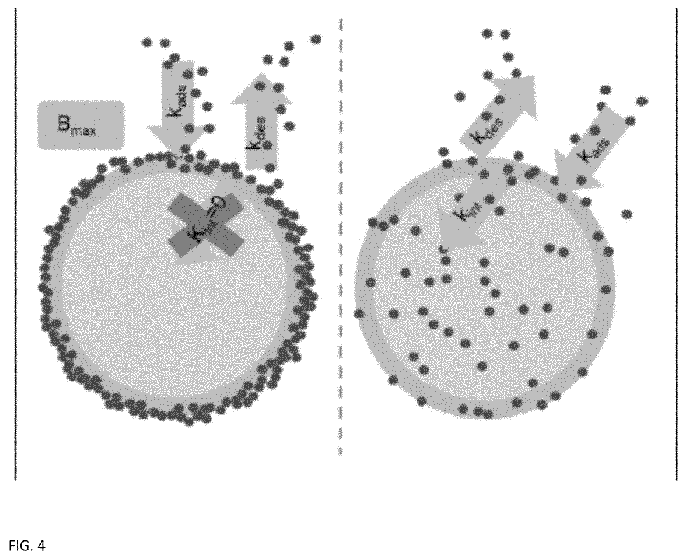

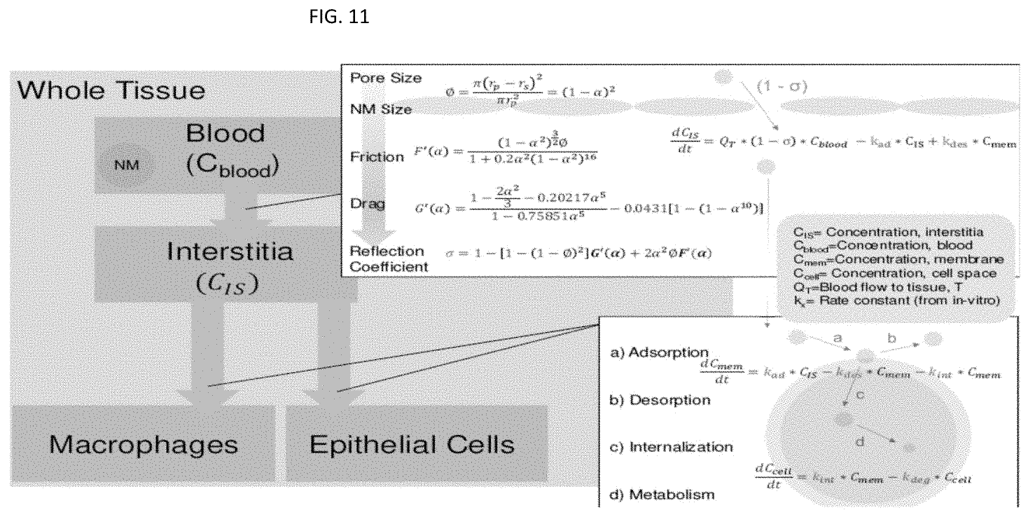

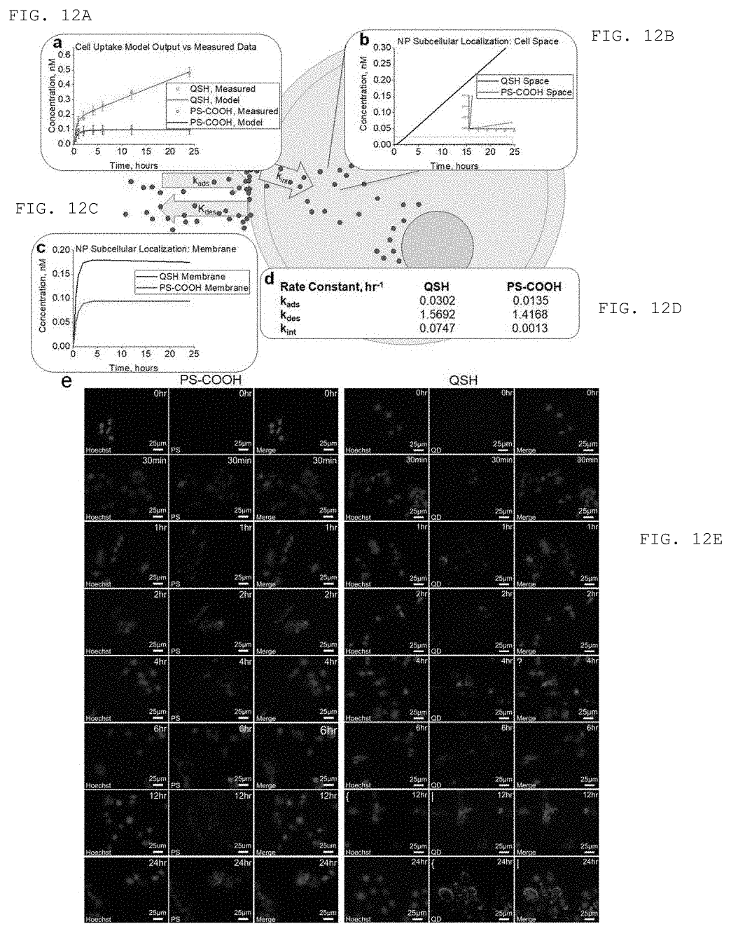

In one embodiment, multiple cell kinetic models are constructed in the MATLAB interface and consist of numerous series of cell-specific first order ordinary differential equations (ODEs) built to be descriptive of basic cellular kinetics with NMs. The series of equations all interact with each-other through basic mass transfer kinetics via first-order rate constants as shown in FIGS. 4 and 11. Overall, the models consist of a media compartment, equation 5, and tissue-specific cell membrane and space compartments, equations 6 and 7, respectively:

$$\begin{matrix}\frac{dMedia}{dt} = - {k}_{ad} Media + {k}_{des} Mem & ( 5 ) \\ \frac{dMem}{dt} = {k}_{ad} Media ( {B}_{ma x} - Mem ) - {k}_{des} Mem - {k}_{int} Mem & ( 6 ) \\ \frac{dSpace}{dt} = {k}_{int} Mem & ( 7 )\end{matrix}$$

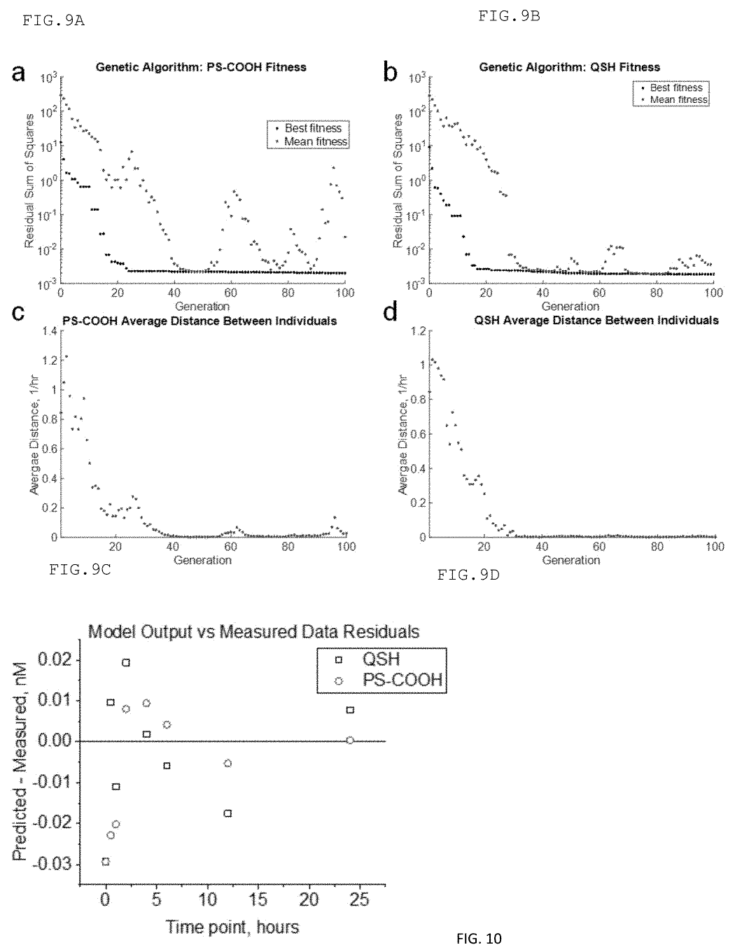

The media (Media), membrane (Mem), and cell space (Space) compartments all interact with each other through mass transfer kinetics driven by rate constants for adhesion (k_{ad}), desorption (k_{des}), and internalization (kᵢₙₜ). Binding capacity (Bₘₐₓ) is used to govern cell adsorption capacity during the NM-cell interaction process. Equations are fit to datasets. ATP-inhibition cell kinetic datasets are used to optimize the parameters for maximum binding capacities, adsorption, and desorption, Bₘₐₓ, k_{ad}, and k_{des} respectively. For this situation, internalization (kᵢₙₜ) is set to 0, mimicking experimental conditions. Parameter estimation is typically conducted using MATLAB version 2015b and 2017b with the Genetic Algorithm included in the statistical package. Above equations were fit to measured RCF datasets through optimization of model parameters kads, kdes, kint. Optimization was performed through the genetic algorithm (GA) included in the optimization toolbox in MATLAB. Originally developed by Holland in the early 1970s, the GA provides a robust optimization of parameters based on evolutionary ideas of natural selection. For optimization, initial vectors (chromosomes) comprised of rate constants (genes) were randomly populated by the GA, fed to the cell kinetic model, calculated for fitness, underwent selection, crossover, and mutations to maximize diversity and produce better fitness at each iteration (generation). At model convergence (20-50 generations, FIG. 9), the optimized parameters provided reasonable correlation coefficients (R=0.9944 and 0.9380 at P<0.001), coefficients of determination (R2=0.9889 and 0.8798), standard errors (S=0.0152 and 0.0158 nM), and residuals for carboxy-coated QD (QSH) and polystyrene (PS-COOH) respectively (Table 3 and FIG. 10).

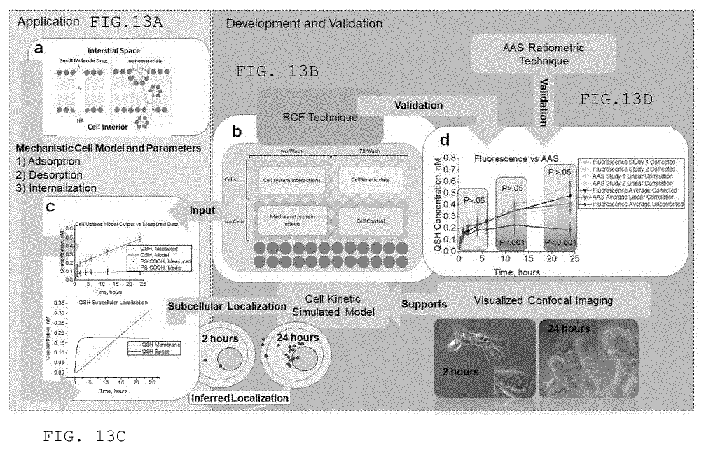

Visuals of model outputs show reasonable fit to measured RCF data for both NM types (FIG. 12a-e). Differing profiles from FIG. 12a indicate minimal saturable uptake for QSH and maximum saturation at 1-2 hours for PS-COOH. Saturability was further studied via subcellular (membrane and cell space) analysis for both NM types (FIG. 12 b, c). QSH membrane quantities were initially higher, until 12 hours, where internalized quantities dominated the total cell space. PS-COOH maintained high membrane binding with minimal internalization for all time points. Rate constants (FIG. 12d) indicated that membrane binding and internalization are the rate limiting steps for QSH and PS-COOH, respectively. Remarkably, confocal microscopy (FIG. 12e) analysis supports this evidence indicating saturable uptake for PS-COOH at 2 hours and no saturable uptake for QSH. Supporting simulation results for QSH, images from FIG. 12e also shows QSH localization around the nucleus inside the cell at 24 hours, indicative of substantial internalized quantities. Thus, confocal images are in agreement with and support the simulation results taken from the validated RCF datasets.

TABLE 3
Model Output Statistics to Measured Datasets

| | | | | | Residual |
|---|---|---|---|---|---|
| | | | | | Sum of |
| | | P- | R | Std. Error, | Squares, |
| Simulation Type | R | value | Square | nM | nM |
| QSH Model | 0.9944 | 0.0000 | 0.9889 | 0.0152 | 0.0019 |
| PS-COOH Model | 0.9380 | 0.0006 | 0.8798 | 0.0158 | 0.0020 |

### In Vivo Simulation Model

The in-vivo model includes organ types that are typically represented in a full physiologically based pharmacokinetic (PBPK) model: kidney, liver, spleen, lungs, heart, small intestine, large intestine, stomach, skin, brain, bone, and pancreas, see FIG. 5. Each tissue has its own weight, as well as total blood flow rate (Qx, where x=tissue type). Specifically, an arterial flow rate towards the tissue and a venous flow rate leaving the particular tissue. New parameters in this model reflects accurate biological and mechanistic assumptions for NM disposition. Accumulation in these tissues are due to two factors, 1) fenestrations, or gaps, in endothelial cells and 2) tissue cell-NM affinity. Overall, these fenestrations can act as paracellular pathways for NM transport into tissues. To this end, each tissue is compartmentalized into cellular-level architecture. Epithelial tissue cells and macrophages (EC), interstitial fluid (IS), endothelial cells, and lymphatic vessels (LV) all make up an entire tissue compartment and reflect accurate physiological properties, FIG. 5. It is here that all kinetic data for tissues and NM are incorporated.

Cellular level interactions with NMs is reflected through tissue specific rate constants optimized to in vitro datasets from the in vitro model as discussed above. For this task, NM disposition through the intravenous (IV) route was captured. This eliminates the need to account for intestinal absorption and enzymatic degradation that may be incident on the NM during the absorption process. All NM dose is expected to be fully absorbed into the blood supply at 0 hours. In this case, it is expected that the particular NM travels from the arterial blood supply towards the tissues according to their tissue-specific blood flow rate, or Qx. Once in the tissue compartment, the NM can leave the blood supply one of two ways: paracellular or transcellular. In the model, all paracellular transport is defined through tissue and NM size specific reflection coefficients (σ). This allows for only a specific fraction of NM to pass into the next compartment, given the size of the pores in tissues and NM diameter. Reflection coefficients are smaller in value for tissues with large pores or sinusoids (e.g. liver, pancreas) and larger for tissues with minimal fenestrae (e.g. brain, kidney). Regions that contain reflection coefficients include 1) blood supply-interstitia and 2) lymphatic vessel-interstitia interfaces. Transcellular transport will be primarily accounted for with the endothelial cells themselves in the blood vessel lining. Once inside the interstitial space, the NM can interact with the epithelial or macrophage cell membrane compartment through adsorption, desorption, or internalization (optimized kinetic rates from in vitro model). Otherwise, it may also get taken up through the lymphatic vessels through the aforementioned reflection coefficient. Once inside the lymph vessels, the NM is expected to drain to a central lymph node compartment (LN) and back into the blood supply. Clearance of NM is also important to capture. The NM is expected to be cleared out through liver (hepatic) clearance into the biliary system.

FIG. 11 provides a diagram showing the transfer of NM from blood vessels to the interstitial and the equations involved in calculating this effect. FIG. 11 also shows the transfer from the interstitial to macrophages and epithelial cells. This involves a) adsorption, b) desorption, c) internalization and d) metabolism. According to FIG. 11, NM transport to cells of a particular tissue is captured through the PBPK simulation. In order for a nanomaterial to reach target tissue cells, it must first bypass the endothelial cell lining primarily through paracellular transport. Here, intercellular gaps are represented by a predicted reflection coefficient, σ. The predicted reflection coefficient builds on previous mechanistic studies by Bungay and Brenner, Lightfoot et al., and Lewellen where hydrodynamic transport is captured, which includes steric exclusion (ϕ), hindrances to diffusion, drag, and pressure drop across the NM (G′(α)), and frictional interactions with the wall (F′(α)). Inclusion of these factors now afford the flexibility to predictively calculate for the reflection coefficient based on variable NM and pore size, rather than providing an empirical constant based on two-pore formalisms obtained from a non-variable single nanomaterial of fixed size that PBPK models currently use. Once the nanomaterial travels from the blood (C_{blood}) through paracellular transport to interstitial space (C_{IS}), it can then interact with the macrophages or epithelial cells (Cₘₑₘ, C_{cell}) of that particular tissue through rate constants (kₓ) determined from the in-vitro RCF assay and cell kinetic model. All compartments herein are described as a series of differential equations designed to solve for concentrations (CO utilizing the MATLAB ODE solver. All simulations were performed in MATLAB v2015b with ODE solver. For optimization of rate constants, matlab function fminsearch was utilized.

### EXAMPLES

### Overview of RCF Method

FIG. 13 shows a schematic overview of the overall scope for RCF method development and application. (a) Shows a schematic of the mechanisms of cellular internalization underlying the basis of the theory of cellular uptake used quantified in (b) and simulated in (c). Here, (a) shows traditional partition coefficients (kp) commonly used for small molecule partitioning between blood supply and cell interior. For NMs, rate constants for adsorption (kad), desorption (kde), and internalization (kint) more accurately represent uptake processes incident on a NM when exposed to cell environment. Upper left region of (b) is descriptive of cellular degradation and interactions with QDs. Upper right, descriptive of fluorescence after washing cells exposed to QDs. Bottom left, descriptive of biological and/or media effects on QD. Bottom right, descriptive of control with cells with no QD exposure. Technique was then (d) validated using a ratiometric AAS approach. (c) Shows a simulation built from the proposed fluorometric datasets with parameters descriptive of adsorption, desorption, and internalization pathways. The model outputs are descriptive of NM localization within a cell and validated against visual confocal imaging.

### Example 1: In-Vitro Quantitation of Cellular Uptake

Tissue cell uptake was captured through a fluorescence assay that is comprised of 4 compartments: the cell kinetic data (CKD, NM exposure to cells with 2× wash and trypsinization), cell system interactions (CSI, NM exposure to cells but no wash), media and protein effects (MPE, NM present but no cells and no wash), and cell control (CC, cells in media and trypsin, no NM added). From this design, quantitative concentrations of NM interacting with cells in-vitro are obtained ratiometrically. A significant difference between MPE and CSI compartments suggests cell or media induced degradation exists, which we found to be present with QD for all cell lines. Validation of cellular degradation is discussed below. Raw signals (I_{CKD}) were calibrated for these effects through ratiometric analysis. These results are entered into a cell kinetics simulation to obtain rate constants of NM-cell interaction that are translatable to our whole-body (in-vivo) simulation, see below.

$${[ Uptake ]}_{c , t} = \frac{{I}_{{CKD}_{t}}}{{I}_{{CSI}_{t}}} * [ Dose ]$$

The fluorescence assay was built starting from the application of cells on a 96-well plate in 4 “compartments”: the Cell System Interactions (CSI) compartment (cells+NM (unwashed), accounts for cell-induced NM degradation), the Cell Kinetic Data (CKD) compartment (cells+NM (washed), measurement of NM uptake), and the Cell Control (CC) compartment (cells in media+no NM (unwashed), control with untreated cells to subtract background signal). The Media and Protein Effect (MPE) compartment (no cells+NM in media (unwashed)) accounts for media and protein induced degradative effects on the NM in the absence of exposure to cells. Note that the CSI and MPE compartments are never washed and therefore maintain the initial applied dose of NM (10 nM). The CKD compartment is washed at each time, t, to remove NMs that are not cell membrane bound or internalized by cells. Control experiments on blank wells showed minimal NM adhesion to the sides and surface of wells, indicating all fluorescence should strictly come from NM interacting with cells. For assay development and validation to AAS for QD, we used 18 wells per compartment, which resulted in one 96-well plate per time point. For the application of this assay to cell types for rate extraction used in in-vivo simulations, we applied QD at a dose of 10 nM to each compartment in triplicate. At time of application, we allowed cells to reach 90% confluence and establish membrane integrity (48 hours). At time zero, the CSI, CKD, and MPE compartments were dosed with 10 nM QSH or PS (10% FBS DMEM suspension), with one NM type per plate. Comparing (by t-test) the fluorescence signal for wells in the CSI compartment at time t with the fluorescence signal from wells in the MPE compartment at time t gives insight into cell-induced degradation. If they are statistically different, we conclude cell induced NM degradation is present and the quantity of fluorescence signal loss due to this effect is determined from the difference of CSI and MPE at time t. Similarly, comparing the fluorescence signal (by t-test) from wells in the MPE compartment at time t with respect to MPE at time zero gives a description of media-induced degradation. These critical steps guide NM uptake calculations, especially if degradation is present.

### Example 2: In-Vitro Cell Kinetic Simulation

The overall simulation is a 3-compartment model in which the laws of mass transfer kinetics are utilized.

Media Compartment.

This compartment includes the media environment from which cells receive their respective NM dose. The initial dose condition was taken as the 10 nM applied in the fluorescence assay. The media compartment NM dose evolution with time is then described as:

$$\frac{d [ Med ]}{dt} = - {k}_{ads} * [ Med ] + {k}_{des} * [ Mem ] \qquad ( 2 )$$

Where [Med] is concentration (nM) of NM in media, [Mem] is the concentration (nM) of NM adhered to cell membrane, and k_{ads}, k_{des} are the first-order rate constants for adsorption and desorption to and from the cell membrane, respectively.

Cell Membrane Compartment.

The cell membrane compartment includes the outer portion of the cell with which the NM interacts. This compartment separates the media from the internal space of the cell. NMs that are internalized by the cell must first adsorb to this compartment through the adsorption rate constant, k_{ads}. Once adsorbed, NMs can 1) leave this compartment through desorption, kdes or 2) enter the cell via kᵢₙₜ as expressed by:

$$\frac{d [ Mem ]}{dt} = {k}_{ads} * [ Med ] - {k}_{des} * [ Mem ] - {k}_{int} * [ Mem ] \qquad ( 3 )$$

with parameters described above.

Cell Space Compartment.

The cell space compartment receives NMs that have transported inside the cell via the first order rate constant for internalization (kᵢₙₜ). Here, NMs can become degraded if the process occurs (determined through the fluorescence assay). The cell space compartment NM dose evolution as a function of time is then described as:

$$\frac{d [ Cell ]}{dt} = {k}_{int} * [ Mem ] - {k}_{deg} * [ Cell ] \qquad ( 4 )$$

where [Cell] is the concentration (nM) of NM in cell interior at time t and k_{deg} is the first order rate constant for degradation of QD obtained from optimization to raw datasets (see below). The degradation rate constants were used to track quantities of NM degraded over the course of the experiment.

### Example 3: In-Vitro Rate Constant Optimization

Here, the genetic algorithm (GA), originally developed by Holland in the early 1970s was used as an artificial intelligence algorithm for a robust optimization of parameters based on evolutionary ideas of natural selection. For optimization, initial vectors (“chromosomes”) comprised of rate constants (“genes”) were randomly populated by the GA, fed to the cell kinetic model, calculated for fitness, underwent selection, crossover, and mutations to maximize diversity and produce better fitness at each iteration (“generation”). Next, we reconsidered QD raw concentration values containing degradation effects to determine the rate of degradation (k_{deg}), holding the previously optimized adsorption, desorption, and internalization rates (k_{ads}, k_{des}, and kᵢₙₜ) as constant. All simulations were performed in MATLAB v2015b. Parameter optimization was implemented with the genetic algorithm (GA) optimization function from the Optimization Toolbox. Parameters for estimation included:
Initial Population: 300
Population Size: 50
Generations: 100
Mutation Rate: Mutation Gaussian
Crossover Rate: 0.80
Selection Function: Stochastic Uniform
   The genetic algorithm was evaluated using the residual sum squares as the fitness function, equation below:

$$RSS = {\sum }_{i}^{n} {( {y}_{i} - {m}_{i} )}^{2}$$

where RSS represents the residual sum of squares from model output (mᵢ) at time (i) to measured data (yᵢ) for n time points. Standard error was computed as

$$S = \sqrt{\frac{RSS}{n}}$$

where S is standard error, RSS residual sum of squares, and n is total time points.
Model output upper and lower bounds were evaluated at the 95% confidence interval through CL (95%)=mᵢ±2*S
where CL(95%) represents 95% confidence limit. The GA was run for 100 generations, enough to allow for convergence at a fitness value representative of measured data.

### Example 4: Physiologically-Based Pharmacokinetic Model Simulation

Nanomaterial transport to cells of a particular tissue is captured through the whole-body NM PBPK simulation. In order for a nanomaterial to reach target tissue cells, it must first bypass the endothelial cell lining primarily through paracellular transport. Here, intercellular gaps are represented by a reflection coefficient, σᵥ. The reflection coefficient builds on previous studies by Bungay and Brenner, Lightfoot et al., and Lewellen where hydrodynamic transport is captured, which includes steric exclusion (ϕ), hindrances to diffusion, drag, and pressure drop across the sphere (G′(α)), and frictional interactions with the wall (F′(α)). The NM must travel from the blood vasculature (Cᵥ) to the interstitial space (C_{IS}) through equation

$$\frac{{dC}_{v}}{dt} = \frac{{Q}_{t} \cdot {C}_{{LU}_{V}}}{{V}_{v}} - \frac{( {Q}_{t} - {Q}_{L} ) \cdot {C}_{V}}{{V}_{V}} - ( 1 - {σ}_{V} ) - {k}_{{ad}_{endo}} \cdot {C}_{V} + \frac{( {k}_{{des}_{endo}} \cdot {A}_{mem} )}{{V}_{V}}$$

Where the tissue blood flow (Qₜ) and lung NM concentration C_{LUV} serve as inputs to this compartment. The NM will interact with the endothelial cell membrane (Aₘₑₘ) of that tissue's compartment via the adsorption (k_{adendo}) and desorption (k_{desendo}) rate constants determined from in-vitro data. The vasculature reflection coefficient (σ_{V}) serves as guidance for the NM to enter the interstitial space given by

$$\frac{{dC}_{IS}}{dt} = ( 1 - {σ}_{V} ) \cdot \frac{{Q}_{L} \cdot {C}_{V}}{{V}_{IS}} - {k}_{ad} \cdot {C}_{IS} + {k}_{des} \cdot {C}_{mem}$$

Where the individual tissue cells (epithelial and macrophages) will interact with the NM through the pre-determined in-vitro rate constants k_{ad} and k_{des} for their cell membranes (Cₘₑₘ). The flow rate into the interstitial space is set to the lymphatic flow rate and vasculature interstitial volumes (V_{IS}) guide the concentration for this compartment. Once the NM enters the tissue cell membrane compartment,

$$\frac{{dC}_{mem}}{dt} = {k}_{ad} \cdot {C}_{IS} - {k}_{int} \cdot {C}_{mem} - {k}_{des} \cdot {C}_{mem}$$

it will desorb via the desorption rate constant (k_{des}) or be internalized into the cell space via the internalization rate constant (kᵢₙₜ). Once inside the cell space, the NM can be thus degraded or sequestered within the cellular environment (C_{cell}).

$$\frac{{dC}_{cell}}{dt} = {k}_{int} \cdot {C}_{mem} - {k}_{deg} \cdot {C}_{cell}$$

All tissue compartments herein are described as a series of differential equations designed to solve for concentrations utilizing the MATLAB ODE solver.

A NM located within tissues can accumulate inside the interstitia, vasculature, or within variable cell types. To account for accumulation within variable cell types in this complex architecture, in-vitro cellular kinetics were translated to the in-silico animal simulation where each tissue compartment contains 4 sub-compartments (epithelial, endothelial, interstitial, and macrophage). In order for a NM to transport to the interstitia of a certain tissue, it was assumed that it travels through the fenestrations unique to the capillaries of that particular tissue each with variable sizes found in literature. These fenestrations were represented by reflection coefficients (σᵥ) computed herein to include the effects of particle drag and frictional hindranceakin to that of a sphere through an artificial porous membrane. The calculated vascular reflection coefficient was then held as a constant in a series of ordinary differential equations representing mass transfer kinetics from the blood supply to interstitial space where the NM will react with tissue cells through first rate constants optimized to our in-vitro data.

Due to the liver and spleen being common targets for NM sequestration, we used these tissues as case-studies to understand and capture the sensitivity and accuracy of the simulation's tissue and cellular outputs to changes in NM size. A 33 and 83% increase in particle size will produce an increase of 15 and 37% uptake in total liver tissue. When compared to observed data, this lies within 2-fold error and follows the same observed positive correlative trends between size and uptake. This phenomenon can be explained through the fact that as NM size increases, such that it exceeds other tissue pore sizes, the NMs from those tissues should then funnel towards tissues with larger pores—in this case, liver. Variable (20-500 nm) diameter biodistribution analysis using Chen 2015 datasets support this principle. Upon reaching the target tissue, the NM will interact with cells through rate constants determined in-vitro. Liver endothelial cells have direct exposure to the blood supply, and thus have immediate interaction with the NMs at the tissue site, which reach peak uptake capacity at approximately 24 hours (Alalaiwe study). As the NM enters the interstitia of the tissue, it quickly interacts with epithelial (liver, Alalaiwe study) and macrophage (spleen, Chen study) tissue cells through our in-vitro rate constants. To validate overall model quantification at the single cell level, we used splenic macrophage uptake data from the Chen study (Chen, K. H.; Lundy, D. J.; Toh, E. K.; Chen, C. H.; Shih, C.; Chen, P.; Chang, H. C.; Lai, J. J.; Stayton, P. S.; Hoffman, A. S.; Hsieh, P. C., Nanoparticle distribution during systemic inflammation is size-dependent and organ-specific. Nanoscale 2015, 7 (38), 15863-72). When using the in-vitro rate constants calibrated for degradative effects, all simulations show accurate predictions (<2-fold error) for NMs ranging from 2-500 nm in individual macrophages when compared to observed harvested splenic macrophages. If simulations used rate constants optimized to raw datasets, all simulation predictions would severely under-predict NM uptake in macrophages, leading to >2-fold error for most outputs. These results build confidence in the predictive power of the simulation and capability to predict variable-sized drug content at the single cell level for animals for the first time, strictly from in-vitro data.

### Example 5: Rate Constant Scaling from In Vitro Data to In Vivo Tissue

No current body simulation scales rate constants obtained for nanomaterials from in-vitro data to account for the number of cells present in each tissue of an organ in the body. The only rate constant scaling that has been reported is for macrophages. The capability to scale in-vitro rate constants for tissue cells from in-vitro to whole organs in body simulation has not been contemplated before this disclosure.

In order for rate constants for nanomaterials obtained in-vitro to translate to in-vivo body simulations, they must be scaled appropriately to account for the number of cells present in each tissue of an organ in the body. It is important to scale according to 1) species and 2) cell populations in a particular tissue in-vivo that are relevant to the uptake studies performed in-vitro. General scaling from RCF datasets are shown in equation 1 below:

$${k}_{scaled} = {k}_{vitro} * ( \frac{1}{{N}_{{cells}_{well}}} ) * {N}_{{cells}_{tissue}}$$

where kscaled is the scaled rate constant (1/hr/tissue), kvitro is the in-vitro rate constant (1/hr/well), Ncellswell is the number of cells per well (cells/well), and Ncellstissue is the number of cells per tissue being scaled to (cells/tissue).

The scaling of these rate constants from in-vitro to whole-body simulations provides the ability to translate data obtained from cell-work to a complete understanding of rates of interactions with cells of tissues in living humans and/or animals.

### Example 6: Determining Metabolism of NM in the Cell and Tissue Microenvironment

Metabolism (degradation) of nanoparticles is currently an unknown phenomena, and currently there exists no validated technique to quantify this occurrence. Currently, nanoparticle inhibition of enzyme metabolism is being quantified¹, but the effects of the metabolic environment on the nanoparticles that are susceptible to degradation has been minimally studied^{2,3}. These studies have given insight into enzymatic degradation, but are unable to translate to applications for others to further understand degradation in the body. In-vivo studies do hint that nanoparticle metabolism is predominant (in liver) and shown to accumulate in the Kuepfer cells as well as endothelial cells⁴.

The in-vitro RCF approach gives quantitative insight on cell-induced metabolism. If present, the degradation rate constant can be calculated from the dataset that includes this metabolic phenomena. This approach provides the capability to 1) understand whether cells will metabolize a known nanomaterial and 2) provide a quantitative rate at which the nanomaterial will be metabolized that can 3) translate to body simulations. This approach can be applied to any material where the fluorescence is a direct measure of the integrity of the system. It is believed that extraction of a metabolic rate constant from the RCF approach based on fluorescence has not been previously contemplated.

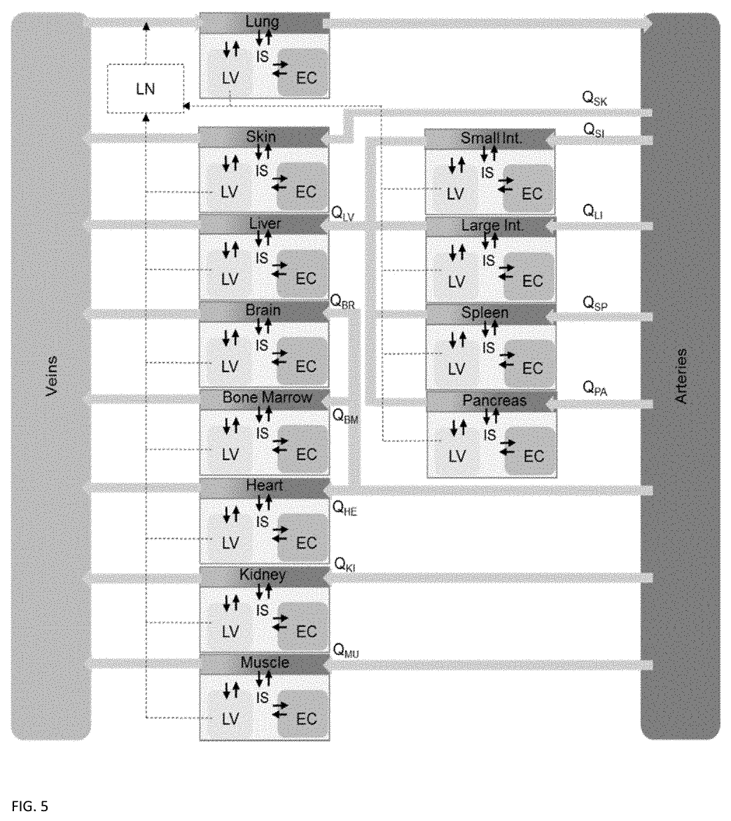

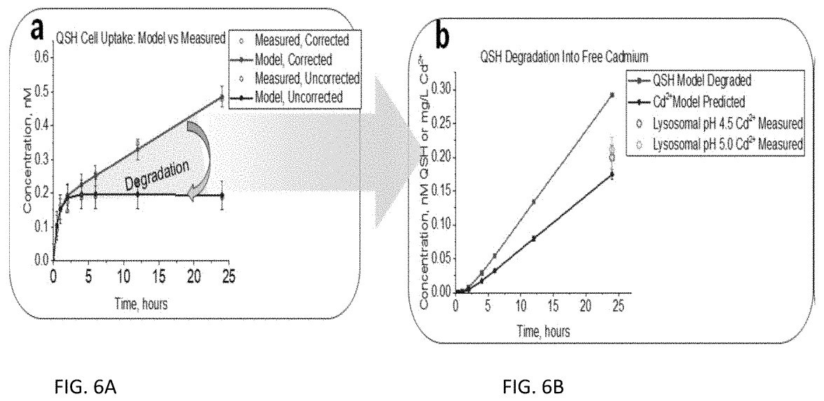

In order to obtain this metabolic rate, rate constants kads, kdes, and kint, which reflect adsorption, desorption, and internalization must first be optimized to the corrected data with no metabolism present. Once obtained, the metabolic rate constant is then optimized to fit the simulation to measured RCF data that includes metabolism. The metabolic rate constant gives insight into how much material is being degraded or metabolized inside the cell (see FIG. 6). Here, the red and blue lines in a show simulated fits to data without and with metabolism (respectively) described above. Next, FIG. 6b shows the predicted nanoparticle metabolized quantities (FIG. 6b, red line) and its representative cadmium concentration (FIG. 6b, blue line).

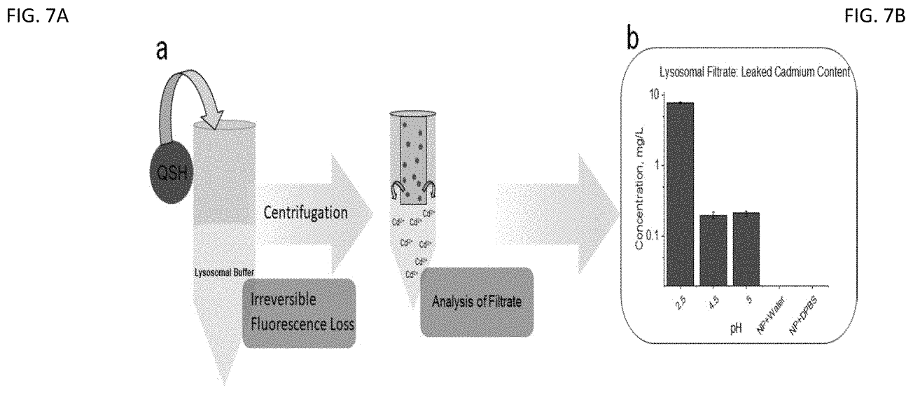

The predicted metabolized cadmium concentrations (FIG. 6b, blue line) were validated against measured in lysosomal cadmium content (FIG. 6b, pink and turquoise) through exposure to model systems of cellular lysosomal environment (see FIG. 7). Predicted simulated metabolized quantities match measured quantities in lysosomal environment. Also, the calculations for determining transport into the cell is provided on the lower portion of FIG. 11.

### REFS FOR EXAMPLE 6

1. Ollikainen, E.; Liu, D.; Kallio, A.; Makila, E.; Zhang, H.; Salonen, J.; Santos, H. A.; Sikanen, T. M., The impact of porous silicon nanoparticles on human cytochrome P450 metabolism in human liver microsomes in vitro. European journal of pharmaceutical sciences: official journal of the European Federation for Pharmaceutical Sciences 2017, 104, 124-132.
2. Akagi, T.; Higashi, M.; Kaneko, T.; Kida, T.; Akashi, M., Hydrolytic and Enzymatic Degradation of Nanoparticles Based on Amphiphilic Poly(c-glutamic acid)-graft-L-Phenylalanine Copolymers. Biomacromolecules 2006, 7, 297-303.
3. Gu, J.; Xu, H.; Han, Y.; Dai, W.; Hao, W.; Wang, C.; Gu, N.; Xu, H.; Cao, J., The internalization pathway, metabolic fate and biological effect of superparamagnetic iron oxide nanoparticles in the macrophage-like RAW264.7 cell. Science China. Life sciences 2011, 54 (9), 793-805.
4. Briley-Saebo, K.; Bjornerud, A.; Grant, D.; Ahlstrom, H.; Berg, T.; Kindberg, G. M., Hepatic cellular distribution and degradation of iron oxide nanoparticles following single intravenous injection in rats: implications for magnetic resonance imaging. Cell and tissue research 2004, 316 (3), 315-23.

### Example 7: Determining Transport of NM from Blood Supply to Tissue Cells

The transport of a nanoparticle from the blood supply to the tissue cells is similar to that of the characteristics of a particle through an artificial porous membrane, originally proposed by Pappenheimer¹. This can be simulated through fluid dynamic theory, primarily obtained from a simulation of spheres through pores captured through a reflection coefficient, a, originally derived and validated by Curry in 1974². This validated approach is folded in the body simulation to provide a flexible, predictive, and truly mechanistic method of transport of any nanoparticle from the blood supply to the tissues.

Currently, no nanoparticle simulation to date calculates for a nanoparticle's transport through pores in the vasculature based on size of the nanoparticle and pores in vasculature. Instead, current techniques use a constant, fixed, and estimated value originally fitted to measured animal data many years ago^{3,4,5}. This current approach severely limits the flexibility and predictive capabilities, as fixed values can only work for 1 particular material with a permanent size. Thus, any nanomaterial with varying diameters cannot be simulated accurately and mechanistically. Additionally, all current reflection coefficients are fixed for all organs and assume there are only two types of pores in all tissues of the body: large and small. This assumption can be greatly improved, as pore sizes range in each organ, as well as across organs.

Given our approach, transport from blood to tissue or lymph for nanoparticle with a variable size can be predicted based on real physics of the body. This will give valuable insight into how effective a current or novel nanomaterial may be in going from blood to tissue. Additionally, the provided technique can help provide data for those interested in understanding how variations in tissue capillary pores can affect transport of their particular material into target or non-target tissues of the body e.g. normal versus cancerous or diseased state.

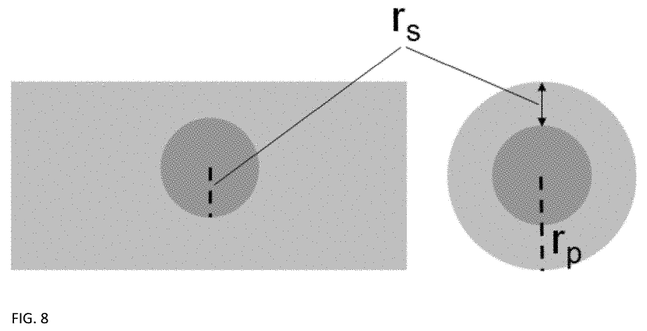

In theory, a nanoparticle must travel through a vasculature pore to enter the interstitial space of a particular tissue. Upon encountering a pore in the vasculature, a nanoparticle cannot occupy positions smaller than 1 nanoparticle radius from the pore's edge in blood vessel fenestrae (see FIG. 8). Nanoparticle entry through pores become sterically restricted as the nanoparticle approaches the size of the pore⁶. This effect is described by the solute partition coefficient below (see also FIG. 11):

$$∅ = \frac{{π ( {r}_{p} - {r}_{s} )}^{2}}{π {r}_{p}^{2}} = {( 1 - α )}^{2}$$

$$α = \frac{{r}_{s}}{{r}_{p}}$$

Where α is the ratio of solute radius to pore radius. The partition coefficient above (Ø) is the ratio of the area available to the solute to the total pore surface area, accounting for the steric hindrance the particle has upon entering the pores of the fenestrae in blood vessels. However, the particle experiences a frictional hindrance from the pore walls upon entering. The frictional hindrance factor F(α) is a factor that defines this phenomena, capturing reduction in diffusion due to hindrance that the wall exerts on the particle through the viscosity of the fluid and is captured through equation below:

$${F}^{′} ( α ) = \frac{{( 1 - {α}^{2} )}^{\frac{3}{2}} ∅}{1 + 0.2 {{α}^{2} ( 1 - {α}^{2} )}^{16}}$$

The drag force, or velocity of the sphere relative to the maximum velocity of water when the sphere is being carried along by the water, is captured through the hydrodynamic function G′(α) according to equation below:

$${G}^{′} ( α ) = \frac{1 - \frac{2 {α}^{2}}{3} - 0.20217 {α}^{5}}{1 - 0.75851 {α}^{5}} - 0.0431 [ 1 - ( 1 - {α}^{10} ) ]$$

The reflection coefficient accounts for the hydrodynamics of convection and diffusion of hard spheres within a right cylindrical pore. The reflection coefficient is independent of the number of channels. When the reflection coefficient approaches 0, the nanoparticle enters the pathways in the membrane. When it is approaching 1, the pore excludes the nanoparticle and it remains outside the pores. The equation for the reflection coefficient accounts for both the frictional force and drag, equation below:

σ=1−[1−(1−Ø)²]G′(α)+2α²ØF′(α)

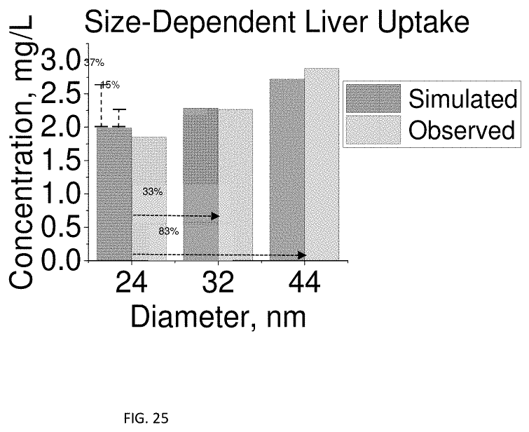

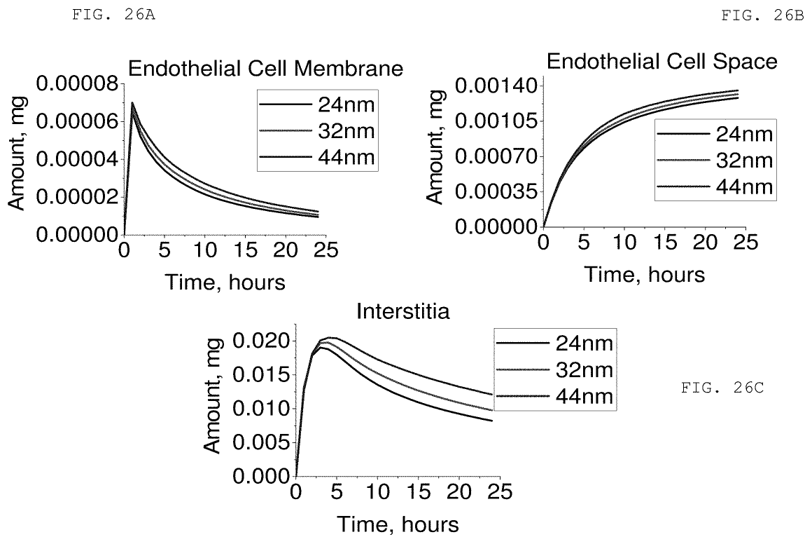

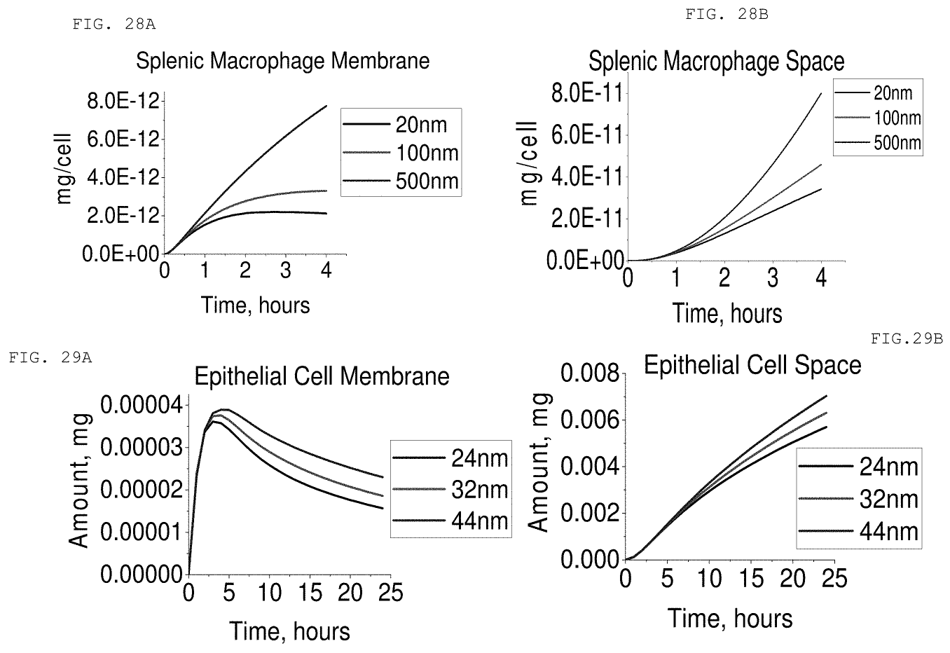

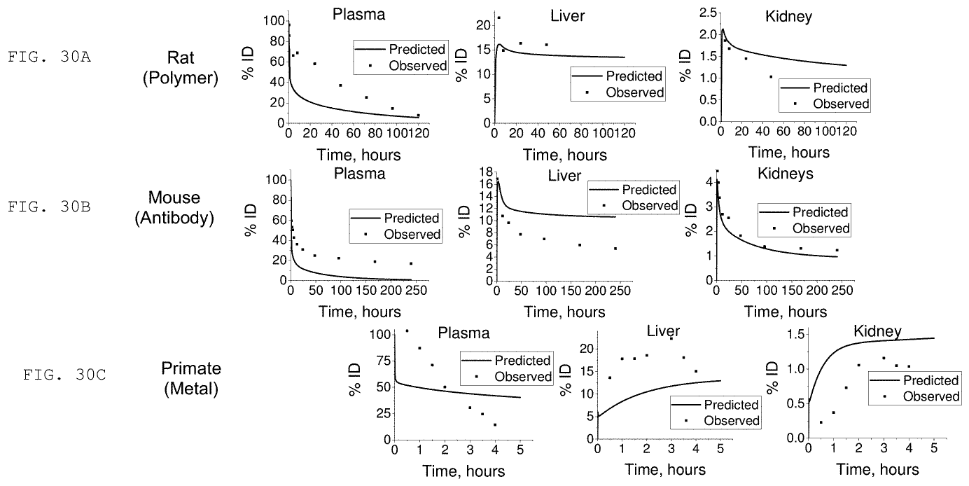

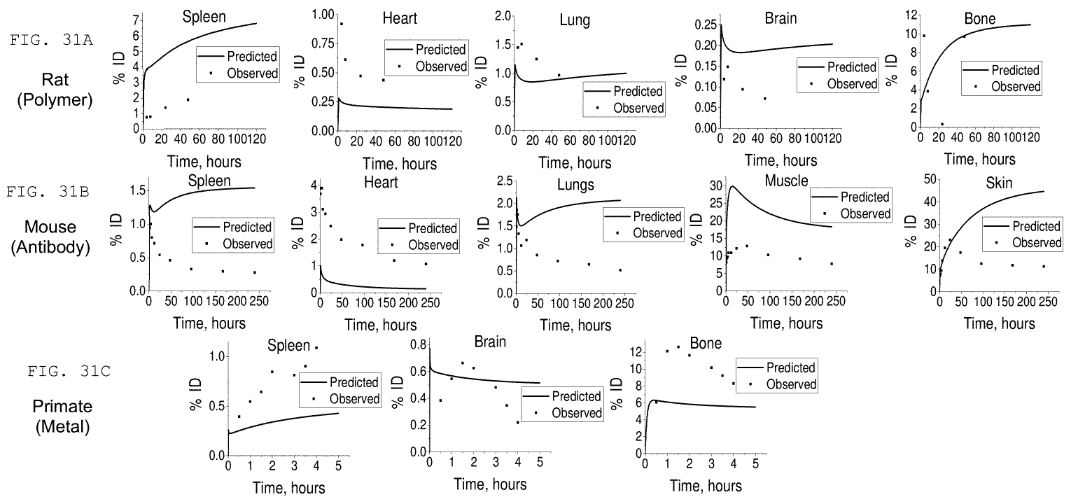

The above equation for the reflection coefficient builds on previous studies including Bungay and Brenner, Lightfoot et al., and of Lewellen where they accounted for full hydrodynamics including steric exclusion, hindrances to diffusion, drag, and pressure drop across the sphere, torque and rotation produced by viscous interactions with the wall. These studies all validated the assumptions to measured data for permeability across animal tissue. This formula assumes no interactions between solute particles. Also, the relationship of pore size, NM size, Friction and drag and the calculations involving determination of the reflection coefficient are set forth in the top part of FIG. 11. Also, FIGS. 25-29 show the sensitivity size on NM transport and uptake.

Inclusion of the now calculated vasculature reflection coefficient based on pore and nanomaterial radius, drag, and friction into a fully functional nanomaterial body simulation predicts cellular level quantities of transport and uptake in all tissues. It is believed that factoring these considerations has never heretofore been contemplated.

### REFS FOR EXAMPLE 7

1. Pappenheimer, J. R.; Renkin, E. M.; Borrero, L. M., Filtration, Diffusion and Molecular Sieving Through Peripheral Capillary Membranes. Capillary Permeability 1951, 167.
2. Curry, F. E., A Hydrodynamic Description of the Osmotic Reflection Coefficient with Application to the Pore Theory of Transcapillary Exchange. Microvascular Research 1974, 8, 236-252.
3. Garg, A.; Balthasar, J. P., Physiologically-based pharmacokinetic (PBPK) model to predict IgG tissue kinetics in wild-type and FcRn-knockout mice. Journal of pharmacokinetics and pharmacodynamics 2007, 34 (5), 687-709.
4. Baxter, L. T.; Zhu, H.; Mackensen, D. G.; Jain, R. k., Physiologically Based Pharmacokinetic Model for Specific and Nonspecific Monoclonal Antibodies and Fragments in Normal Tissues and Human Tumor Xenografts in Nude Mice. Cancer Research 1994, 54, 1517-1528.
5. Covell, D. G.; Barbet, J.; Holton, O. D.; Black, C. D. V.; Parker, R. J.; Weinstein, J. N., Pharmacokinetics of Monoclonal Immunoglobulin d, F(ab′)2, and Fab′ in Mice. Cancer Research 1986, 46, 3969-3978.
6. Bassingthwaighte, J. B., A practical extension of hydrodynamic theory of porous transport for hydrophilic solutes. Microcirculation 2006, 13 (2), 111-8.

### Example 8: In-Vitro Quantitation of Degradation

Signals obtained from RCF assay include:
1. I_{CSI,0}
2. I_{CSI,t}
3. I_{MPE,0}
4. I_{MPE,t}
5. I_{CKDt}
6. I_{CC}

Overall, raw fluorescence descriptive of cell uptake (I_{CKD,t}) was taken relative to raw fluorescence of unwashed cells at time t (I_{CSI,t}) to obtain a calibrated fraction of uptake (f_{cell,c}):

$${f}_{cell , c} = \frac{{I}_{{CKD}_{t}}}{{I}_{{CSI}_{t}}} \qquad ( 1 )$$

Raw fluorescence descriptive of cell uptake (I_{CKD,t}) was also taken relative to raw fluorescence of unwashed cells at time 0 (I_{CSI,0}) to obtain a raw fraction of uptake (f_{cell,r}):

$${f}_{cell , r} = \frac{{I}_{{CKD}_{t}}}{{I}_{{CSI}_{0}}} \qquad ( 2 )$$

These two fractions were then used to obtain concentration of NM uptake using the general equation,

[Uptake]_{c,t}=f_{cell,x}*[Dose] (3)

where f_{cell,x} is the fraction of uptake for x=raw or corrected, [Uptake]ₜ is the concentration of NM taken up by cells (nM), and [Dose] is the applied dose in nM. To determine if cell-induced degradation is present, a two-tailed t-test was performed between unwashed CSI and MPE compartments at time, t, (I_{CSI}_{ₜ} and I_{MPE}_{ₜ}, respectively). To determine if media-induced degradation is present, a two-tailed t-test was performed between unwashed MPE at time 0 and time t. Cell-induced degradation (I_{cdeg}_{ₜ}) was taken as the difference between unwashed without (I_{MPE}_{ₜ}) and with (I_{CSI}_{ₜ}) cell exposure,

I_{cdeg}_{ₜ}=I_{MPE}_{ₜ}−I_{CSI}_{ₜ} (4)

If media degradation was present, the intensity of this degradation type was taken as the difference between unwashed wells without cell exposure from time 0 to time t.

I_{mdeg}_{ₜ}=I_{MPE}_{₀}−I_{MPE}_{ₜ} (5)

Taken together, the sum of these values equals the total degradation that a NM can undergo for the RCF assay (I_{deg}_{ₜ}):

I_{deg}_{ₜ}=I_{cdeg}_{ₜ}+I_{mdeg}_{ₜ} (6)

### Example 9: Assessment of NM Toxicity

MTS (CellTiter 96 AQ Non-Radioactive Cell Proliferation, VWR) assay was run to determine toxicity of a variety of NMs at different doses (QSH and PS) for optimal NM exposure conditions. NMs were applied to murine Hepal-6 cells for a period of 24 hours. Briefly, cells were seeded in triplicate onto wells of a clear flat bottom 96-well plates at a density of 34,700 cells/well and left 24 hours for attachment. At time, media was aspirated, and 100 uL of all NM solutions were applied to wells, except controls, for a period of 24 hours in 37° c. CO₂ incubator. Negative controls were kept in media to retain complete viability and positive controls were kept in water for cell death. All NMs were diluted in DMEM supplemented with 10% FBS at various doses, ranging from 5 nM to approximately 250 nM. At time, cells were washed 2× with complete growth medium and re-applied with 100 uL of DMEM with 10% FBS. 20 uL aliquots of MTS was added and background absorbance was captured at 490 nm. Plates were then incubated for 2 hours and absorbance checked again. Sample absorption values were normalized to that of cells exposed to complete growth medium.

### Example 10: AAS Quantification of Cd Content within Cells

Samples were collected from all compartments in the fluorescence assay (as discussed in Example 1) at each time t, degraded equally in 33% v/v aqua regia (AR), and measured for absolute cadmium content. AAS measurements were referenced to a 6-point cadmium calibration curve constructed with equal % v/v AR, as well as lied above LOD and LOQ.

### Example 11: Lysosomal Colocalization Analysis

Cells were seeded onto 35 mm diameter tissue coated petri dishes (35 mm TC-treated culture dish, Corning) with 2 mL of 347,000 cells/mL solution and left in incubator at 37° C. and 5% CO₂ for 24 hours. Cells were washed 1× with complete growth medium and 2 mL of 10 nM QSH solutions were added. After 24 hours, petri dishes were removed from incubator and washed 2× with complete growth media. Lysotracker Green (DND-26, ThermoFisher Scientific) was added at 1 uM concentration and confocal images obtained. Lysosomal colocalization studies were performed using a spinning-disk confocal imaging system. Z-stacks were taken at 2 um step sizes, with a total distance of 40 um.

### Example 12: Cell Kinetic Confocal Microscopy

A paralleled series of cell kinetic samples containing QSH were analyzed for uptake using confocal microscopy. At each time point in the study, cells were washed 2×, trypsinized, and transferred to 35 mm petri dishes containing 2 mL of complete growth medium. After 24 hours, cells were washed 2× with complete growth medium, and Hoechst33257 was applied. Cells were then imaged for NM uptake with 20 2 um step sizes.

### Example 13: Simulated Lysosomal Buffer Analysis

Cellular lysosomal environment was mimicked to determine stress induced on fluorescence through lysosomal material exposure. The citric acid (, >99.5%, ACS Reagent, Sigma-Aldrich) simulated lysosome chelator buffer at pH 2.5-5.0 was created and used as the solvent for QSH and PS. Controls contained pH 7.4 DPBS buffer solutions. More specifically, stock solutions of 0.25 and 0.19 mM solutions of sodium citrate monobasic (Anhydrous, Sigma-Aldrich) and dibasic (Sesquihydrate, Sigma-Aldrich), respectively, were made. Stock solutions of 50 mM and 20 mM citric acid stock solution were made in separate vials as well. Then 6 solutions of equal concentrations of 10 nM QSH were made in either sodium citrate monobasic/dibasic with citric acid. To achieve desired pH of 2.5, 3.0 3.5, 4.5, or 5.0. pH was adjusted by combination of dibasic or monobasic sodium citrate stock solution with small aliquots of citric acid solutions. For size analysis, Zetasizer (Malvern) DLS measurements were obtained. Here, samples were diluted in-situ in solvents of desired pH and measurements obtained immediately after. Fluorescent plate readings were run in triplicates of 100 uL of solutions applied to wells of a 96-well plate system. Fluorescence was taken with 580 or 525 excitation and 595 or 620 nm emission, respectively for QSH or PS, using a Tecan M200 plate reader. To check for Cd²⁺ core leakage, 10 nM QSH and PS were analyzed for fluorescence in PBS, water, and simulated lysosomal buffer at pH 2.5, 4.5, and 5.0 at 0 hours and 24 hours exposure. For each time point, samples were collected and centrifuged at 15,000×g for 20 minutes through an Amicon Ultra 10 kDa filter to separate possible cations from QSH. Filtrate was then analyzed for free cadmium content using a PerkinElmer atomic absorption spectrometer with a cadmium hollow cathode lamp with wavelength of 288.65 nm. Flow rate was adjusted to 4 mL/min and samples were run in triplicate.

### Example 14: Prolonged Cell Exposure Analysis

Prolonged exposure to intracellular environment analysis was performed after washing at time, t. Here, QSH or PS washed samples at time, t, were left to incubate to an additional 12-x and 24-x hours, where x is the time of wash for each particular sample. At total experimental time of 12 and 24 hours, previously washed plates were mixed and measured for fluorescence changes from their original time, t. An example is shown below:

2 hours wash fluorescence→10 hour post wash (12-2 hours) fluorescence→22 hours post wash (24-2 hours) fluorescence
Importantly, 12 hour washed sample only contained 24-x prolonged cell exposure data and 24 hour washed sample contains no prolonged exposure, given that cell exposure was only allowed for the duration of a total time of 24 hours.

### Example 15: Calculation of Fluorescent Plate Reader Limits of Detection and Quantitation

The limits of detection (LOD) and limits of quantitation (LOQ) were calculated from construction of an 8-point calibration curve with concentration ranging from 0.10-10 or 15 nM for QSH or PS. The LOD and LOQ were calculated based on the standard deviation of the response signal of the blank and slope of the linear curve through zero, equations below:

$$\begin{matrix}LOD = \frac{3 σ}{S} & ( 8 ) \\ LOQ = \frac{10 σ}{S} & ( 9 )\end{matrix}$$

where σ is the standard deviation of the blank (NM suspension in trypsin) and S is the slope of the calibration curve. All readings were performed on a TecanM200 Pro.

### Example 16: Examples of NMs Analyzed in Animal Studies for Simulation

The metal-based particles comprised of PEG2000 (32 nm) or 5000-coated Au (28 nm) and PEG2000 (33 nm) or PHEA (66 nm) coated SPIO NMs. Polymer based NMs consisted of PAA-PEG2000 (35 nm), PLGA (197 nm), PGA (112 nm), and PS-PEO (107 nm). QD studies varied substantially, as we analyzed hydroxide (CdSeS—SiOH, 21 nm), mercaptoundecanoic acid (CdSe/ZnS-LM, 25 nm), mercaptosuccinic acid (CdTe/CdS-MSA, 3.8 nm), and mercaptoproprionic acid (CdTe-MPA, 4 nm) coated NMs.

### Example 17: In-Vitro Statistical Analysis

Statistical analysis was performed on Microsoft Excel 2010. All calculated statistical evaluations were performed using the student's two-tailed t-test at the P<0.05, P<0.01, or P<0.001 level.

### Example 18: Tissue-Level Uptake of NMs can be Predicted for Multiple Species

To build confidence in the simulation's predictive power and translational capabilities for different classes of NMs and species of animals, predictions were compared against measured tissue-level content for multiple NM types and species. Complete sets of physiological values (tissue volumes and blood flow rates) were obtained for three species including rats, mice, and non-human primates (NHP) in order to scale cellular content (epithelial, endothelial, macrophage). The NM animal simulation was validated to 15 pre-clinical datasets, which included different dosing scenarios (0.029-64.3 mg/kg BW), NM types (polymer, QD, metal, and antibody), and NM sizes (4-197 nm diameter). As most laboratory biodistribution data only captures total uptake at the tissue level, validation of our simulation included total tissue (sum of macrophage, epithelial, endothelial, vascular, and interstitial) content. Evaluations of predictive performance were performed according to world health organization guidelines as well as standards accepted by pharmaceutical and academic consortiums involved in drug development and safety. Specifically, 49.78, 33.31, and 16.90% of datasets lied within <2-fold, <3-fold, and >3-fold error respectively, demonstrating reasonable model performance. Antibodies exhibited the highest percentage of distributions lying within 2-fold error, followed by metal, polymer, and QD-based NMs.

Although assumptions of spherical morphology are not quintessential for antibodies, this assumption was practical enough for high model predictivity for this NM. Just like other materials in the nanometer range (˜10 nm), their distribution properties are also limited by biological membranes, so size, charge, and molecular weight will affect their biodistribution. Although no metabolic clearance mechanism is included in the simulations yet, 50% of IgG plasma outputs lied within 2-fold, 42% within 3-fold and 8% greater than 3-fold error, suggesting good model predictivity. Plasma outputs generally underpredicted uptake, most likely due to lack of IgG-endothelial FcRn receptor binding, which future in-vitro assays could capture. Simulation outputs were slightly overestimated for tissues with high FcRn expression where IgG catabolism is prominent (skin, muscle, and liver). IgG catabolism would be a necessary improvement in order to correct the simulation over estimation for these tissues (1.70, 2.30, and 1.42 fold-error over estimate).

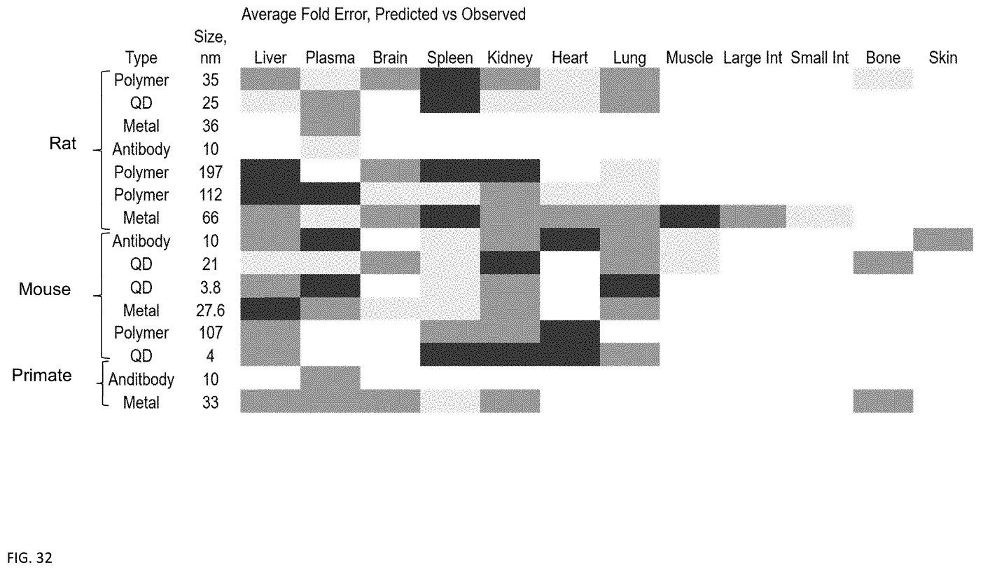

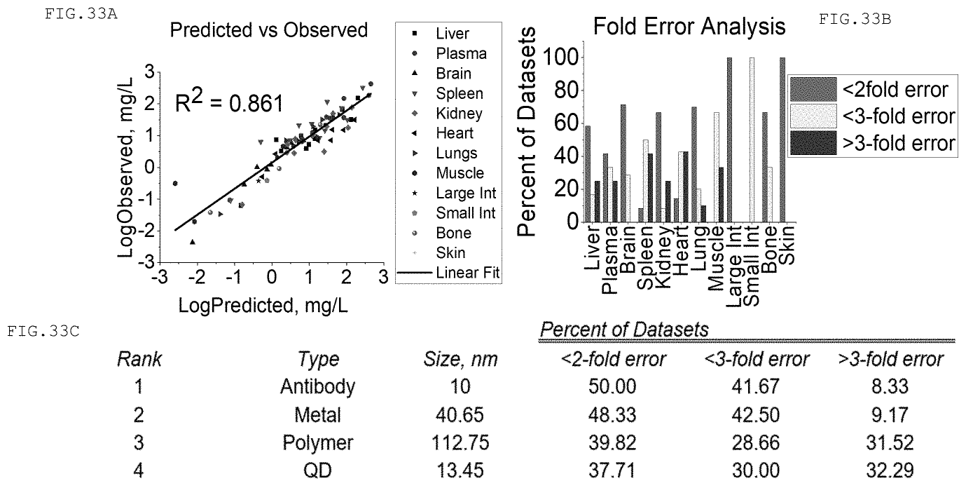

Substantial analysis was performed in major reticuloendothelial system (RES) tissues (liver and spleen), each with differing uptake profiles and relative quantities, dependent on tissue macrophage content. Overall, simulations mostly fell within 2-fold error of observed values for many of these studies, but generally underpredict total tissue content primarily due to transcellular transport not being accounted for in our simulation. This effect is prominent for simulation outputs for the 25 nm CdSe/ZnS QD (liver) and 4 nm CdTe-MPA QD datasets for a variety of tissues. The 25 nm CdSe/ZnS-LM QDs showed accurate (<2-fold error) liver tissue predictions when compared to observed data from live animal studies, with a larger proportion of uptake in macrophages relative to epithelial cells, as both simulation and literature electron microscopy evidence shows. In this case, observed data from these studies positively correlates NM accumulation within Kupffer cells to minimal biliary or fecal excretion, demonstrating the significance and balance between macrophage and epithelial cell interactions with NMs in living systems. The spleen, with approximately 10× more macrophage content than the liver, exhibited relatively higher percentage macrophage uptake than liver tissue for 28 nm PEG5000-Au (mice) 33 nm PEG2000-SPIO (NHP), 66 nm PHEA-SPIO, 35 nm PEG2000-PAA, and 106 nm PS-PEO NM types. See FIGS. 30-33 for results of experiments relating to average fold error for predicted vs observed values of different NM materials.

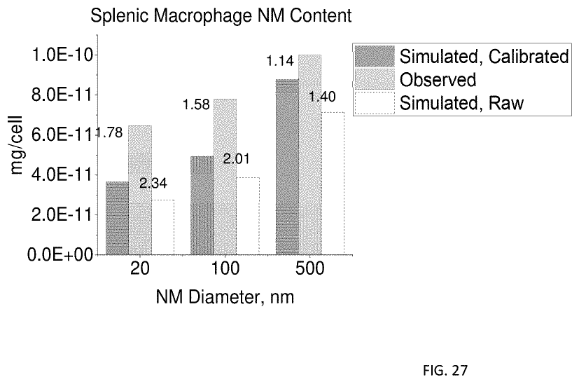

Overall, NMs between 10 and 100 nm diameter (n=10) exhibited the best predictive capabilities for our simulation (>50% below 2-fold error), most likely due to the fact that 1) cellular rate constants obtained in-vitro translated well to the live animal simulation, 2) paracellular transport through endothelial fenestrations was the primary means of tissue transport, and 3) biliary and/or renal clearance of NM minimally affected biodistribution. Approximately 50% of datasets of NMs with diameters <10 nm lied above 3-fold error and 40% lie below 2-fold error. For NMs >100 nm in diameter (n=3), the simulation demonstrated similar errors within all error ranges (36.43, 25.71, and 37.86%<2, <3, and >3-fold error respectively). Here, we suspect NM size to exceed pore diameter, limiting tissue uptake, so transcellular transport would be necessary to fully capture tissue uptake. Simulated and observed datasets were compared through log-analysis of the averages of the datasets and plotted against each other, as shown in FIG. 27. Model predicted averages and observed averages were fairly linearly correlated, indicative of reasonable model predictivity across all species and NM types (R²=0.861).

It should be borne in mind that all patents, patent applications, patent publications, technical publications, scientific publications, and other references referenced herein and in the accompanying appendices are hereby incorporated by reference in this application to the extent not inconsistent with the teachings herein.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

1. A method comprising
obtaining fluorescence measurements of a first group of containers comprising a population of cells exposed to a dose of nanomaterial (NM) (unwashed (CSI)); a second group of containers comprising a population of cells exposed to a dose of the NM (washed and disrupted (CKD)); a third group of containers comprising a dose of the NM without cells (MPE); and a fourth group of containers comprising a control population of cells without a dose of the NM and disrupted (CC); and
determining rate kinetics of the NM based on comparing the fluorescence measurements of the first, second, third, and fourth groups.

2. The method of claim 1, wherein disrupted cells comprises trypsinizing the cells.

3. The method of claim 1, wherein determining rate kinetics utilizes a Genetic Algorithm to extract rate kinetics from the fluorescence measurements.

4. The method of claim 1, wherein the first, second, third and/or fourth groups of containers comprise subgroups of containers wherein each subgroup may comprise different cell types.

5. The method of claim 4, wherein the different cell type comprises liver cells, endothelial cells, skin cells, brain cells bone marrow cells, heart cells, kidney cells, muscle cells, epithelial cells, white blood cells, small intestine cells, large intestine cells spleen cells, pancreas cells or lung cells.

6. The method of claim 5, further comprising determining in vivo biodistribution of the NM based on the rate kinetics.

7. The method of claim 1, further comprising quantifying degradation of the fluorescent measurements.

8. The method of claim 7, wherein quantifying degradation comprises:
a. obtaining a calibrated fraction of uptake (f_{cell,c}) according to the following formula:
$${f}_{cell , c} = \frac{{I}_{{CKD}_{t}}}{{I}_{{CSI}_{t}}}$$
wherein (I_{CKD,t}) is raw fluorescence of cell uptake and (I_{CSI,t}) is raw fluorescence of unwashed cells at time t;
b. obtaining a raw fraction of uptake (f_{cell,r}) according to formula
$${f}_{cell , r} = \frac{{I}_{{CKD}_{t}}}{{I}_{{CSI}_{0}}}$$
wherein (I_{CSI,0}) is raw fluorescence of unwashed cells at time 0;
c. determining concentration of NM uptake according to formula:
[Uptake]_{c,t}=f_{cell,x}*[Dose]
wherein f_{cell,x} is f the fraction of uptake for x=raw or calibrated, [Uptake]ₜ is the concentration of NM taken up by cells (nM), and [Dose] is the applied dose in nM;
d. determining cell-induced degradation (I_{cdeg}_{ₜ}) at time t of NM according to the following formula:
I_{cdeg}_{ₜ}=I_{MPE}_{ₜ}−I_{CSI}_{ₜ}
wherein (I_{MPE}_{ₜ}) is unwashed containers without cell exposure and (I_{CSI}_{ₜ}) is unwashed containers with cell exposure;
e. determining media induced degradation I_{mdeg}_{ₜ} at time 0 and time t according to the following formula:
I_{mdeg}_{ₜ}=I_{MPE}_{₀}−I_{MPE}_{ₜ}; and
f. determining total degradation (I_{deg}_{ₜ}) according to the following formula:
I_{deg}_{ₜ}=I_{cdeg}_{ₜ}+I_{mdeg}_{ₜ}.

9. The method of claim 3, wherein determining rate kinetics utilizes the Genetic Algorithm from MATLAB®.

10. A method for determining transport of a NM from blood supply to tissue cells comprising:
a) Calculating a solute partition coefficient Ø according to the following formula:
$$∅ = \frac{{π ( {r}_{p} - {r}_{s} )}^{2}}{π {r}_{p}^{2}} = {( 1 - α )}^{2}$$
$$α = \frac{{r}_{s}}{{r}_{p}}$$
b) Calculating Frictional hindrance F(α) according to the following formula:
$${F}^{′} ( α ) = \frac{{( 1 - {α}^{2} )}^{\frac{3}{2}} ∅}{1 + 0.2 {{α}^{2} ( 1 - {α}^{2} )}^{16}} ;$$
c) Calculating drag force G′(α) according to the following formula:
$${G}^{′} ( α ) = \frac{1 - \frac{2 {α}^{2}}{3} - 0.20217 {α}^{5}}{1 - 0.75851 {α}^{5}} - 0.0431 [ 1 - ( 1 - {α}^{10} ) ] ;$$
and
d) determining a reflection coefficient based on solute partition, frictional hindrance and drag by calculating reflection coefficient by applying the foregoing to the following formula:
σ=1−[1−(1−Ø)²]G′(α)+2α²ØF′(α)

11. The method of claim 10, further comprising determining transport of NM from cellular interstitial to tissue cell membranes by
$$\frac{{dC}_{mem}}{dt} = {k}_{ad} * {C}_{IS} - {k}_{des} * {C}_{mem} - {k}_{int} * {C}_{mem}$$

12. The method of claim 10, further comprising determining transfer of NM from the blood supply to tissue interstitia by making the following calculation:
$$\frac{{dC}_{IS}}{dt} = {Q}_{T} * ( 1 - σ ) * {C}_{blood} - {k}_{ad} * {C}_{IS} + {k}_{des} * {C}_{mem}$$

13. The method of claim 10, further comprising determining transfer of NM into cells by making the following calculation:
$$\frac{{dC}_{cell}}{dt} = {k}_{int} * {C}_{mem} - {k}_{deg} * {C}_{cell}$$

14. (canceled)

15. (canceled)

16. A system comprising
a computer programmed to predict concentration of an administered nanomaterial (NM) in an in vivo tissue of an animal species based on rate constants optimized to fluorescence measurements of a first group of containers comprising a population of cells exposed to a dose of NM (unwashed (CSI)); a second group of containers comprising a population of cells exposed to a dose of the NM (washed and trypsinized (CKD)); a third group of containers comprising a dose of the NM without cells (MPE); and a fourth group of containers comprising a control population of cells without a dose of the NM and trypsinized (CC), wherein the population of cells from the first, second and fourth groups includes subgroups of cells of different cell types of the animal species; and a memory component associated with the computer wherein the memory component has stored thereon multiple sets of ODEs (ordinary differential equations) representing mass transfer kinetics of blood flow rate between and inside multiple different tissues of the different cell types.

17. The system of claim 16, wherein the ODEs comprise an ODE representing mass transfer kinetics from blood supply to interstitia.

18. The system of claim 16, wherein the computer is programmed to receive information of NM size and utilize said NM size information to predict the concentration of NM in the in vivo tissue.

19. A computer programmed to conduct the calculations of steps a-d of claim 10.

20. The system of claim 17, wherein the ODEs further comprise an ODE representing mass transfer kinetics from interstitia to macrophages and epithelial cells.

21. The method of claim 1, wherein determining rate constants comprises determining kads, kdes, and kint.

22. The method of claim 21, further comprising determining metabolic rate of the NM, comprising optimizing kads, kdes, and kint to calibrated data with no metabolism present and then establishing a metabolic rate constant that is optimized to fit calibrated data with no metabolism present.
